(19) United States
(12) Patent Application Publication
(10) Pub. No.: US 2011/0293574 A1
(43) Pub. Date: Dec. 1, 2011

(54) Hematopoietic stem cell growth factor

(76) Inventors: John P. Chute, Durham, NC (US); Heather Himburg, Durham, NC (US)

(21) Appl. No.: 12/998,208

(22) PCT Filed: Sep. 28, 2009
(86) PCT No.: PCT/US2009/005347

§ 371 Date: Aug. 12, 2011

Related U.S. Application Data
(60) Provisional application No. 61/100,618, filed on Sep. 26, 2008.

(51) Int. Cl. A61K 35/12; A61K 38/18; A61P 7/00; C12N 5/0789

(52) U.S. Cl. 424/93.7; 435/406; 435/372; 514/7.9

(57) ABSTRACT

The present invention relates, in general, to stem cells and, in particular, to a hematopoietic stem cell (HSC) growth factor and to methods of using same.

This application claims priority from U.S. Provisional Application No. 61/100,618, filed Sep. 26, 2008, which is incorporated herein in its entirety by reference.

This invention was made with government support under Grant No. AI 067798 awarded by the National Institutes of Health. The government has certain rights in the invention.

### TECHNICAL FIELD

The present invention relates, in general, to stem cells and, in particular, to a hematopoietic stem cell (HSC) growth factor and to methods, of using same.

### BACKGROUND

Pleiotrophin (PTN) is an 14 kDa heparin binding growth factor that has pleiotrophic effects. PTN is extensively regulated in embryogenesis and is expressed in vascular tissue and connective tissue and in the nervous system during development. PTN expression is largely down-regulated in the adult and has been shown to be expressed only in osteoblasts, Leydig cells, neuronal cells and adipose tissue in adults. PTN has been shown to be a growth factor for epithelial cells, endothelial cells and fibroblasts in culture. PTN is also a proto-oncogene involved in the transformation of breast cancer cells and melanoma. PTN is not known to have any function in hematopoiesis or in the regulation of HSC fate determinations. (See Deuel et al, Arch. Biochem. Biophys. 397:162 (2002), Gu et al, FEBS Letters 581:382 (2007), Meng et al, Proc. Natl Acad. Sci. USA 97:2603 (2000), Perez-Pinera et al, Proc. Natl. Acad. Sci. USA 103:17795 (2006), Fukuzawa et al, Mol. Cell. Biol. 28:4494 (2008).)

Hematopoietic stem cells (HSCs) possess the unique capacity to self-renew and give rise to all of the mature elements of the blood and immune systems (Zon, Nature 453: 306-13 (2008), Orkin et al, SnapShot: hematopoiesis. Cell 132:712 (2008), Kiel et al, Nat Rev Immunol 8:290-301 (2008)). HSC self-renewal is regulated by both intrinsic and extrinsic signals (Zon, Nature 453: 306-13 (2008), Orkin et al, SnapShot: hematopoiesis. Cell 132:712 (2008), Kiel et al, Nat Rev Immunol 8:290-301 (2008), Varnum-Finney et al. Blood 91:4084-91 (1998), Stier et al, Blood 99:2369-78 (2002), Reya, et al, Nature 423:409-14 (2003), Karlsson et al, J Exp Med 204:467-74 (2007), Zhang et al, Nat Med 12: 240-5 (2006), North et al, Nature 447:1007-11 (2007)), but the mechanisms involved in the control of this process are incompletely understood. Several growth factors have been identified whose action is associated with murine HSC self renewal, including Notch ligands (Varnum-Finney et al. Blood 91:4084-91 (1998), Stier et al, Blood 99:2369-78 (2002)), Wnt 3a (Reya, et al, Nature 423:409-14 (2003)), angiopoietin-like proteins (Zhang et al, Nat Med 12: 240-5 (2006)) and prostaglandin E2 (North et al, Nature 447:1007-11 (2007)). Alternately, co-culture of HSCs with supportive stromal or endothelial cells (Hackney et al, Proc Natl Acad Sci USA 99:13061-6 (2002), Chute et al, Blood 100:4433-9 (2002)) or the enforced expression of the transcription factors, HoxB4 or HoxA9 (Zon, Nature 453: 306-13 (2008), Antonchuk et al, Cell 109:39-45 (2002)), can cause robust expansions of HSCs in culture. However, strategies which require cell co-culture or genetic modification of HSCs are not readily translatable into the clinic (Blank et al, Blood 111:492-503 (2008)). Moreover, despite advances in understanding the biology of HSC self-renewal and differentiation, the identification and development of translatable growth factors capable of inducing HSC regeneration in vivo continues to lag.

HSC transplantation is curative therapy for thousands of individuals with hematologic malignancies on an annual basis. However, the ability to perform HSC transplantation on the much larger number of individuals who are eligible is limited by the rarity of HSCs and the inability to amplify these cells for therapeutic purposes. Hundreds of thousands of individuals undergo chemotherapy and/or radiotherapy for the treatment of cancer annually and the majority of these patients suffer hematologic toxicities due to damage to HSCs and progenitor cells. The identification and characterization of novel growth factors that act to cause the self-renewal and expansion of HSCs in vitro or in vivo would provide the basis for new treatments of such patients and could be used to accelerate recovery from chemotherapy and/or radiotherapy. Potentially, hundreds of thousands of individuals could benefit from such a growth factor(s), as has been seen with the administration of Neupogen (GCSF) and Erythropoietin, which stimulate the recovery of neutrophils and red blood cells, respectively.

The present invention results, at least in part, from studies demonstrating that PTN is a soluble growth factor for HSCs and induces the self-renewal of HSCs.

### SUMMARY OF THE INVENTION

The invention relates generally to stem cells. More specifically, the invention relates to a HSC growth factor and to methods of using same to induce or enhance self renewal and/or expansion of HSCs in vivo and in vitro.

Objects and advantages of the present invention will be clear from the description that follows.

### BRIEF DESCRIPTION OF THE DRAWINGS

FIGS. 1A and 1B. Human brain derived endothelial cells (HUBECs) overexpress PTN. (FIG. 1A) Microarray analysis demonstrated that primary HUBECs from 7 different donors overexpressed PTN compared to non-brain endothelial cells (ECs) (n=8). (FIG. 1B) qRTPCR analysis confirmed that HUBECs overexpressed PTN by 100-1000 fold compared to non-brain ECs.

FIGS. 2A-2E. PTN causes the expansion of HSCs observed in HUBEC cultures. CD34⁻KSL cells were cultured for 7 days with HUBECs plus isotype control antibody (IgG) or HUBECs plus anti-PTN antibody (a-PTN). The progeny of these cultures were transplanted into lethally irradiated mice, along with autologous bone marrow (BM) cells for radioprotection. Treatment with anti-PTN blocked the expansion of HSCs in HUBEC cultures, suggesting that PTN signals the self-renewal and amplification of HSCs in vitro. Shown is a scatter plot of 45.1 donor engraftment at 8 weeks in lethally irradiated recipient mice following transplantation of 10 (FIG. 2A), 30 (FIG. 2B) or 100 (FIG. 2C) cells. The 12 week evaluation point is shown in FIGS. 2D and 2E.

FIGS. 3A-3H. HUBEC culture induces a significant expansion of HSCs capable of myeloid, lymphoid and erythroid differentiation and this expansion is negated completely by treatment with anti-PTN. The in vivo repopulation of donor T cells (FIGS. 3A and 3B) and myeloid cells (FIGS. 3C and 3D) was significantly reduced in the HUBEC cultures treated with anti-PTN, implicating PTN as critical to the expansion of HSCs in culture. The expansion of B cells (FIGS. 3E and 3F) and erythroid cells (FIGS. 3G and 3H) in vivo was also essentially negated via treatment with anti-PTN, further confirming that a multipotent repopulating cell was amplified during HUBEC culture and this amplification was eliminated fully via treatment with anti-PTN.

FIGS. 4A-4D. (FIG. 4A). Phenotype analysis of 34⁻KSL progeny cultured with recombinant human PTN. FIG. 4B. Four week competitive repopulating unit (CRU) data. FIG. 4C. Four week CRU estimates. FIG. 4D. Treatment of HSCs with PTN did not alter the normal multilineage differentiation potential of HSCs.

FIG. 5. cDNA sequence for human PTN.

FIGS. 6A-6H. Treatment with PTN is sufficient to induce LT-HSC expansion. (FIG. 6A) C57Bl6 BM MNCs were collected via cytospin and stained with 25 ng/mL of anti-RPTPβ/ζ-FITC antibody or isotype control antibody. (Top) A representative high power field microscopic image (20×) is shown of RPTPβ/ζ staining of BM MNCs versus isotype control. (Bottom) Flow cytometric analysis confirmed that 89% of BM KSL bells expressed RPTPβ/ζ. (FIG. 6B) BM 34⁻KSL cells (500 cells/well) were plated in liquid suspension culture with 20 ng/mL thrombopoietin, 120 ng/mL SCF, and 50 ng/mL Flt-3 ligand (“TSF”) with and without increasing concentrations (10, 100 and 1000 ng/mL) of PTN×7 days. Fold expansion of total cells, % KSL cells and KSL cell expansion is shown. (Left) The addition of 10 ng/ml and 100 ng/ml PTN to TSF (gray bars) caused significant increases in total cells compared to culture with TSF alone (black bars)(mean±SD, n=3, *P=0.01, **P=0.006). (Middle) The % KSL cells also significantly increased in cultures treated with 10 ng/ml or 100 ng/ml PTN+TSF compared to TSF alone (mean±SD, n=3, *P=0.04, **P=0.004). (Right) The progeny of BM 34⁻KSL cells treated with 10 ng/mL or 100 ng/mL PTN+TSF demonstrated a significant increase in total KSL cells compared to the progeny of TSF alone (mean±SD, n=3,*P=0.005, **P=0.006). All comparisons were one-tailed t tests. (FIG. 6C) Limiting doses (10 cells) of BM 34⁻KSL (CD45. 1⁺) cells or their progeny following 7 day culture with TSF alone or TSF+100 ng/mL PTN were transplanted via tail vein injection into lethally irradiated CD45. 2⁺ recipient mice. Levels of donor-derived CD45. 1⁺ cell engraftment were measured in the peripheral blood (PB) at 12 weeks. Scatter plots show the percentages of total CD45. 1⁺ donor cells and donor-derived B220⁺ (B-lymphoid), Mac-1⁺/Gr-1⁺ (myeloid) and Thy1. 2⁺ (T cell) populations in all mice transplanted with 10 BM 34⁻KSL cells or their progeny following culture (n=8-10 mice per group). Mice transplanted with the progeny of 34⁻KSL cells cultured with TSF+PTN demonstrated >10-fold higher total CD45. 1⁺ cell engraftment (mean±SD, P=0.006) and significantly increased B-lymphoid (P=0.003), myeloid (P=0.03) and T cell engraftment (P=0.006) at 12 weeks compared to mice transplanted with the same dose of day 0 BM 34⁻KSL cells or their progeny following culture with TSF alone (P=0.007, P=0.004, P=0.04, P=0.007, respectively; one tailed t test). Horizontal lines represent the mean engraftment levels for each group. (FIG. 6D) Representative flow cytometric analysis is shown of PB donor-derived (CD45. 1⁺) multilineage engraftment at 12 weeks post-transplant in mice transplanted with 10 BM 34⁻KSL cells vs. mice transplanted with the progeny of 10 BM 34⁻KSL cells following culture with TSF+100 ng/mL PTN. Percentages of total are shown in each quadrant. (FIG. 6E) Limiting dilution analysis was performed in which CD45. 2⁺ mice were lethally irradiated and then transplanted with limiting doses (10, 30 and 100 cells) of CD45. 1⁺ BM 34⁻KSL cells or their progeny following culture with TSF alone or TSF+100 ng/mL PTN. Poisson statistical analysis was performed and plots were obtained to allow estimation of CRU content within each condition (n=8-10 mice transplanted at each dose per condition; n=75 mice total). The plot shows the percentage of recipient mice containing less than 1% CD45. 1⁺ cells in the PB at 12 weeks post-transplantation versus the number of cells injected per mouse. CRU estimates for day 0 BM 34⁻KSL cells (red line), the progeny of BM 34⁻KSL cells post-culture with TSF+PTN (blue line) and the progeny of culture with TSF alone (black line) are shown. (FIG. 6F) Mice transplanted with PTN-treated 34⁻KSL cells (striped bars) demonstrated increased repopulation of CD45. 1⁺ donor-derived cells at 4, 8, 12 and 24 weeks compared to mice transplanted with day 0 34⁻KSL cells (black bars, mean±SEM, n=6-10/group, *P=0.006, *P=0.002, *P=0.006, P=0.05) or the progeny of 34⁻KSL cells cultured with TSF alone (gray bars, mean±SEM, ̂P=0.005, ̂P=0.002, ̂P=0.007, P=0.05, respectively). (FIG. 6G) Secondary competitive repopulating transplant assays were performed using BM harvested from primary mice at 24 weeks following transplant with either day 0 BM 34⁻KSL cells (10 cell dose) or the progeny of 34⁻KSL cells cultured with TSF+PTN versus. TSF alone. At 12 weeks post-transplant into CD45. 2⁺ secondary mice, the mice transplanted with BM from mice in the TSF+PTN group demonstrated significantly higher donor CD45. 1⁺ cell repopulation compared to recipients of BM from mice transplanted with day 0 34⁻KSL cells or their progeny following culture with TSF alone (mean±SEM, n=5-6/group, P=0.003 and P=0.02, respectively; Mann-Whitney test). Horizontal bars represent mean levels of CD45. 1⁺ cell engraftment in the PB. (FIG. 6H) Representative FACS analysis is shown of CD45. 1⁺ cell engraftment and B220⁺, Mac-1⁺/Gr-1⁺ and Thy 1. 2⁺ engraftment at 12 weeks post transplant in secondary mice transplanted with BM from primary mice transplanted with day 0 34⁻KSL cells or their progeny following culture with TSF+PTN.

FIGS. 7A and 7B. PTN induces PI 3-k/Akt signaling in HSCs. (FIG. 7A) BM 34⁻KSL cells were placed in culture with TSF alone or TSF+100 ng/mL PTN in the presence (gray bars) and absence of 1 μM wortmannin (black bars), a PI 3-kinase inhibitor, ×7 days. Treatment of 34⁻KSL cells with TSF+PTN increased total cell (left) and KSL cell expansion (middle) compared to TSF alone (mean±SD, n=3, *P=0.04 and *P=0.04, respectively). Conversely, the progeny of wortmannin+TSF+PTN had a significant reduction in total cell and KSL cell expansion compared to cells treated with TSF+PTN (mean±SD, n=3, ̂P=0.02, ̂P=0.02, respectively). The progeny of BM 34⁻KSL cells cultured with TSF+PTN also demonstrated a significant increase in the percentage of cells with phosphorylated Akt compared to the progeny of TSF alone (right) (mean±SD, n=3, *P=0.03). Conversely, the levels of phosphorylated Akt were significantly reduced in the progeny of TSF+PTN+wortmannin compared to the progeny of TSF+PTN (mean±SD, n=3, ̂P=0.04). (FIG. 7B) BM KSL cells were placed in culture with TSF (black bars) or TSF+100 ng/mL PTN (gray bars)×7 days and KSL cells were then isolated via FACS-sorting at day +7 for qRT-PCR analysis and comparison of gene expression. Treatment with TSF+PTN caused a significant increase in the expression of HES-1 (mean±SD, n=3, *P=0.04) and GFI-1 (mean±SD, n=3, *P=0.005) in KSL cells and a significant decrease in PTEN expression (mean±SD, n=3, *P=0.002) compared to culture with TSF alone. All comparisons were one tailed t tests.

FIG. 8A-8C. PTN induces BM stem and progenitor cell regeneration in vivo. Adult Bl6.SJL mice were irradiated with 700 cGy TBI and subsequently treated with 2 μg PTN or saline daily×7 days via intraperitoneal injection (n=10 mice per group). At day +7, all mice were sacrificed and BM cells were collected and analyzed for stem and progenitor cell content and function. (FIG. 8A) PTN-treated mice demonstrated significantly increased numbers of total BM cells and BM KSL progenitor cells compared to controls (mean±SEM, n=5, * P=0.03 and P=0.04, respectively). (FIG. 8B) Functional assays demonstrated an increased number of BM colony forming cells (CFCs) in the PTN-treated group compared to controls (mean±SEM, n=5, *P=0.004). (FIG. 8C) BM HSC content, as measured by the LTC-IC assay, was 11-fold increased in the PTN-treated mice compared to controls at day +7 following high dose irradiation (mean±SEM, n=4, *P=0.02).

FIGS. 9A and 9B. Gene expression analysis of HUBECs versus non brain ECs. (FIG. 9A) mRNA was isolated from primary HUBECs (n=6, 3 replicates per sample) and non-brain ECs (n=8, 3 replicates per sample). Microarray analysis was performed on each sample and a heat map is shown demonstrating the relative expression of genes within HUBECs and non-brain ECs (red=increased expression, green=decreased expression). Unsupervised hierarchical cluster analysis revealed 1335 genes (red bar region) upregulated in HUBECs compared to non-brain ECs. (FIG. 9B) (Left) Scatter plot of microarray analysis of PTN gene expression in HUBECs versus non-brain ECs (mean 25.1+7.4 vs. 1.0+0.3, n=6-8 samples/group, P=0.001). Horizontal lines represent mean PTN expression in each group. (Middle) PTN expression via qRT-PCR in HUBECs vs. non-brain ECs (mean±SEM, n=2-3 per group, HUBECs1 vs. Coronary, P=0.004; HUBECs1 vs. Pulmonary, P=0.004). (Right) PTN concentrations from ELISA of HUBEC Conditioned Media compared to non-brain EC CM (mean±SEM, n=3, *P=0.04).

FIGS. 10A-10C. PTN signaling is necessary for HUBEC-mediated HSC expansion. BM 34⁻KSL cells (500 cells/well) were placed in culture with TSF and compared with non-contact culture with HUBECs+TSF or HUBECs+TSF+50 μg/mL anti-PTN×7 days. IgG isotype antibody was added to HUBECs+TSF cultures to control for the addition of the anti-PTN antibody in the comparison cultures. (FIG. 10A) A limiting dose (30 cells) of BM 34⁻KSL (CD45. 1⁺) cells or their progeny following 7 day culture with HUBECs+TSF+IgG versus HUBECs+TSF+anti-PTN was transplanted via tail vein injection into lethally irradiated (950 cGy total body) CD45. 2⁺ recipient mice. Levels of donor-derived CD45. 1⁺ cell engraftment were measured in the PB at 12 weeks following transplantation in all mice. Scatter plots show the percentages of total CD45. 1⁺ donor cells and donor-derived B-lymphoid, myeloid, and T cell populations in the PB in all mice transplanted with 30 BM 34⁻KSL cells or their progeny following 7 day culture. Mice transplanted with the progeny of TSF+HUBECs+IgG cultures demonstrated significantly higher total CD45. 1⁺ cell engraftment (P=0.03) and engraftment of B-lymphoid (B-220⁺, P=0.004) and myeloid cells (Mac-1/Gr-1⁺, P=0.01) compared to mice transplanted with the same dose of day 0 BM 34⁻KSL cells (mean±SEM, n=7-10/group). Conversely, mice transplanted with the progeny of BM 34⁻KSL cells cultured with TSF+HUBECs+anti-PTN demonstrated significant reduction in total CD45. 1⁺ cell, B-lymphoid, myeloid, and T cell (Thy 1. 2⁺) engraftment compared to mice transplanted with the progeny of TSF+HUBECs+IgG (mean±SEM, n=7-10/group, P=0.004, P=0.0001, P=0.002, P=0.001, respectively; one tailed t test). (FIG. 10B) Representative flow cytometric analysis is shown of PB donor-derived (CD45. 1⁺) multilineage engraftment at 12 weeks post-transplant in a mouse transplanted with the progeny of TSF+HUBECs+IgG cultures versus a mouse transplanted with the progeny of TSF+HUBECs+anti-PTN. Percentages of total are shown in each quadrant. (FIG. 10C) Inhibition of PTN signaling prevents HUBEC-mediated expansion of LT-HSCs. Donor CD45. 1⁺ cell engraftment over time in mice transplanted with day 0 BM 34⁻KSL cells (30 cell dose) or the progeny of 34⁻KSL cells following culture with HUBECs+TSF or HUBECs+TSF+anti-PTN. Engraftment was persistently higher in mice transplanted with the progeny of 34⁻KSL cells following HUBECs+TSF culture (striped bars) as compared to mice transplanted with the same dose of day 0 34⁻KSL cells (black bars), with significant differences at weeks 8 and 12 (mean±SEM, n=7-10/group, *P=0.004 and *P=0.03, respectively). Mice transplanted with the progeny of 34⁻KSL cells cultured with HUBECs+TSF+anti-PTN (gray bars) demonstrated significantly decreased CD45. 1⁺ cell engraftment at 8, 12 and 24 weeks post-transplant compared to mice transplanted with the progeny of 34⁻KSL cells cultured with HUBECs+TSF (mean±SEM, n=7-10/group, ̂P=0.0001, ̂P=0.002, and ̂P=0.002 for weeks 8, 12, and 24, respectively).

FIG. 11. PTN does not signal through β-catenin. BM 34⁻KSL cells (500-1000 cells/well) from flox-β-catenin mice (gray bars) and βcatenin^{-/-} (LoxP,LoxP;Vav-cre) mice (black bars) were plated in culture with TSF alone or TSF+100 ng/mL PTN×7 days. Cells were analyzed at day 7 for % KSL cells in culture to estimate preservation of hematopoietic progenitor cells in response to PTN treatment. No differences were observed in the amplification of % KSL cells in culture between the flox-β-catenin group and the β-catenin−/− group (means±SD, n=3, *P=0.04 and **P=0.04).

FIGS. 12A and 12B. Thrombopoietin, Stem Cell Factor (SCF), Flt-3 ligand combination is superior to individual cytokines alone when combined with PTN.

### DETAILED DESCRIPTION OF THE INVENTION

Various sources of adult endothelial cells (ECs) are capable of supporting the growth and amplification of murine, baboon and human HSCs in vitro. Detailed comparisons of aortic, renal artery, pulmonary artery, umbilical cord blood vein/artery and brain-derived vessels (Circle of Willis) have revealed that HUBECs produce a soluble activity that is capable of inducing a 1-2 log expansion of human HSCs in short term (7 day) culture. These studies have confirmed that this potent expansion of human HSCs does not require cell-to-cell contact, but is mediated strictly by soluble factors produced by HUBECs. Extensive gene expression analysis using microarray has identified the genes that are overexpressed by multiple sources of HUBECs (n=7-10) compared to non-brain HECs (n=7-10) which were confirmed to not possess this hematopoietic-supportive activity. This subtractive analysis revealed several genes with soluble gene products as candidate growth factors for HSCs. PTN was selected for functional characterization. PTN, which has no annotated function in hematopoiesis, is highly expressed during embryogenesis during which time the definitive onset of hematopoiesis occurs. The studies described in the Example that follows demonstrate that PTN is a novel and important growth factor for HSCs and plays an essential role in regulating hematopoiesis in vivo.

The present invention relates to a method of inducing or enhancing self renewal and/or expansion of HSCs (e.g., mammalian HSCs, preferably human HSCs) using PTN (e.g., recombinant PTN). The invention also relates to therapeutic strategies based on the administration to a mammal (e.g., a human) of PTN or HSCs expanded in vitro using PTN.

PTN suitable for use in the methods of the invention can be isolated from a mammal, including a human, or expressed in and isolated from a heterologous host, such as bacteria, yeast, or cultured cells, including insect or mammalian cells (preferably primate cells, more preferably human cells). Methods for isolating and for expressing and purifying polypeptides are well-known in the art. Preferably, the PTN is mammalian PTN (e.g., GenBank accession number CAA37121, AAB24425 NP_{—}002816, or AAH05916).

The use of native PTN (e.g., human PTN) is preferred, however, a fragment or variant thereof that possesses PTN activity, or fusion protein comprising same, can be used. Fragments and/or variants of PTN, having the activity of PTN, or fusions proteins comprising same, can be substituted for native PTN in any of the above or following embodiments of the invention, without an explicit statement to that effect.

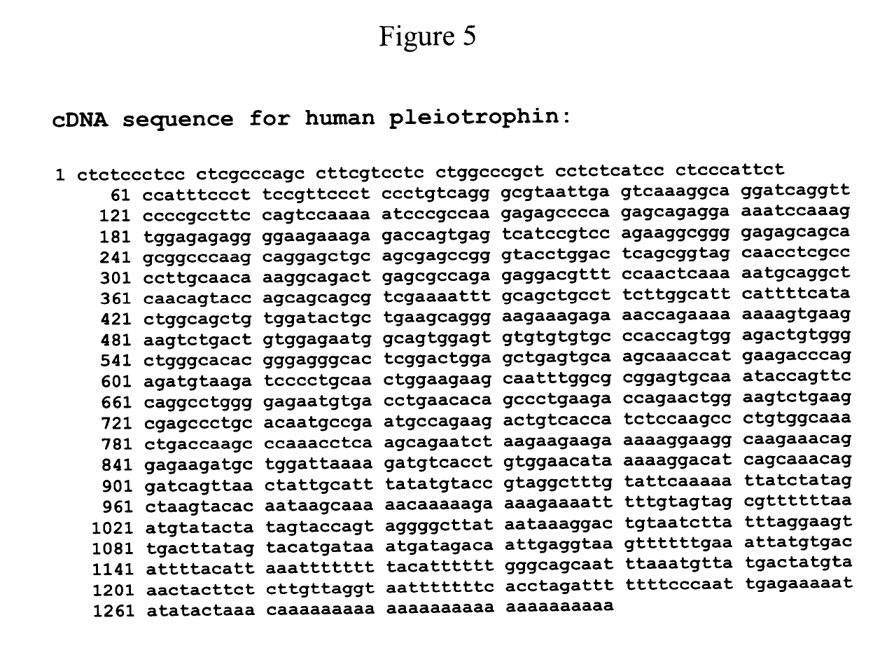

For long term expression, to avoid the need to express, isolate, and/or purify PTN, or to facilitate the expression of PTN in a subset of cells, for example, at the site of delivery, polynucleotides encoding PTN can be used in practicing the methods of the invention. (See FIG. 5.) Such polynucleotides can be present in a vector, such as a viral vector or other expression vector. Viral vectors suitable for use include retrovirus vectors (including lentivirus vectors), adenovirus vectors, adeno-associated virus vectors, herpesvirus vectors, and poxvirus vectors. Other viruses have been shown to be capable of expressing genes-of-interest in cells, and the construction of such recombinant viral vectors is well known in the art. (See, for example, Baum et al, J Hematother 5(4):323-9 (1996); Schwarzenberger et al, Blood 87:472-478 (1996); Nolta et al, Proc. Natl. Acad. Sci. 93:2414-2419 (1996); Maze et al, Proc. Natl. Acad. Sci. 93:206-210 (1996); Mochizuki et al, J Virol 72(11):8873-83 (1998); Ogniben and Haas, Recent Results Cancer Res 144:86-92 (1998).) In addition to viral vectors, non-viral expression vectors can also be used. Any of a variety of eukaryotic expression vectors can be used, provided that expression of PTN in a sufficient quantity (and, as may be appropriate, in an appropriate cell-type-specific manner) is effected. The polynucleotide can be present in the vector in operable linkage with a promoter (e.g., an inducible promoter). Various promoters are known that are induced in HSCs, e.g. IL-2 promoter in T cells, immunoglobulin promoter in B cells, CMV promoter in other cell types, etc. Methods for delivering expression vectors to target cells/tissues include direct naked DNA delivery, liposome-mediated delivery, ballistic DNA delivery, and other means of causing DNA to be taken up by cells. Such methods are well known in the art.

As indicated above, in one embodiment, the invention relates to a method of enhancing proliferation of HSCs in vitro. This method can comprise, for example, culturing HSCs in the presence of an amount of PTN sufficient to enhance proliferation of the HSCs. Advantageously, the HSCs are cultured in the presence of PTN, thrombopoietin, stem cell factor (SCF) and Flt-3 ligand (TSF). (See, for example, optimal concentration determinations in Chute et al, Blood 105:576-583 (2005).)

To effect expansion of HSCs in vitro, the HSCs can be cultured in an appropriate liquid nutrient medium. Various media are commercially available and can be used. Culture in serum-free medium may be preferred. After seeding, the culture medium can be maintained under conventional conditions for growth of mammalian cells.

Populations of HSCs expanded in vitro can be used in transplantation to restore hematopoietic function to autologous or allogeneic recipients (e.g., mammalian recipients, such as humans). For example, the expanded HSCs can be used to accelerate hematologic recovery of patients following chemo- or radiation-therapy. In a specific aspect of this embodiment, marrow samples can be taken from a patient and stem cells in the sample expanded; the expanded HSCs population can serve as a graft for autologous marrow transplantation following chemo- or radio-therapy. Transplantation of the expanded HSCs can be effected using methods known in the art.

For autologous transplantation, HSCs can be expanded ex vivo via culture with PTN, advantageously, in combination with TSF, and the expanded graft can be utilized, for example, for individuals who have suboptimal PB collection in order to facilitate engraftment in the patient. For allogeneic stem cell transplant, PTN can be utilized (advantageously, in combination with TSF), for example, to expand umbilical cord blood cells to facilitate the more rapid engraftment of donor HSCs and engraftment of mature cells in cord blood transplant recipients. Cord blood is an ideal alternative source of donor HSCs for the 50-60% of adult patients who lack an HLA matched donor since incompletely HLA matched CB units can be safely transplanted in patients without a high rate of graft versus host disease; in principle, therefore, CB could become a universal donor source of HSCs for adults who need a stem cell transplant. However, CB transplantation in adults has not become standard of care due to the unacceptably high rate of graft failure and delayed hematologic recovery in adult recipients, leading to unacceptably high rates of infectious mortality. These issues are primarily a function of the relatively small dose of HSCs in each CB unit. Therefore, a method to reliably expand CB HSCs, (e.g., using PTN, advantageously, in combination with TSF), can dramatically improve the potential for CB transplant to be utilized for the large number of patients who are otherwise eligible for a CB transplant in the treatment of their disease.

In another embodiment, the present invention relates to a method of enhancing the proliferation of HSCs (e.g., mammalian HSCs) in vivo. The method is useful for generating expanded populations of HSCs and thus mature blood cell lineages. The method is also useful for facilitating/promoting more rapid hematologic recovery in vivo in patients. This is desirable, for example, where a mammal has suffered a decrease in hematopoietic or mature blood cells as a consequence of, for example, radiation, chemotherapy or disease. The method of the present invention comprises administering to a mammal (e.g., a human) in need thereof PTN in an amount and under conditions such that proliferation of HSCs in the mammal is effected.

One skilled in the art can optimize the amount of PTN to be used in vitro, ex vivo or in vivo. By way of example, about 100 ng/mL can be used in vitro with HSCs in culture with, for example, one exposure at day 0. For in vitro expansion of HSCs, exemplary ranges of TSF components are: thrombopoietin at 20-50 ng/ml, stem cell factor at 100-200 ng/ml, and Flt-3 ligand at 20-50 ng/ml. In vivo, by way of example, about 1 mcg PTN can be administered daily subcutaneously×14 days beginning on day +1 following completion of chemotherapy or radiotherapy. The actual amount of PTN to be administered (e.g., to a human patient) can depend on numerous factors, including the physical condition of the patient and the effect sought.

While the methods of the invention are preferred for use in humans, they can also be practiced in domestic, laboratory or farm animals, such as dogs, horses, cats, cows, mice, rats, rabbits, etc.

Certain aspects of the invention can be described in greater detail in the non-limiting Example that follows. (See also Chute et al, Blood 100:4433-4439 (2002), Chute et al, Blood 105:576-583 (2005), Epub 2004 Sep. 2.)

### Example 1

### Experimental Details

### Antibodies

Recombinant human PTN, goat anti-PTN, and goat IgG were purchased from R&D systems (Minneapolis, Minn.).

### Endothelial Cell Culture

Human endothelial cell lines derived from the following vessels: uterine microvessel, umbilical artery, iliac artery, dermal microvessel, coronary artery, and lung microvessel were obtained from Lonza (Portsmouth, N.H.) and cultured according to the recommended guidelines. Six human brain endothelial cell (HUBEC) lines were derived as previously described (Chute et al, Stem Cells 22:202-215 (2004), Chute et al, Blood 105:576-583 (2005), Chute et al, Blood 100:4433-4439 (2002)) and maintained in complete endothelial cell culture medium containing M199 (GIBCO/BRL, Gaithersburg, Md.), 10% heat-inactivated fetal bovine serum (FBS) (Hyclone, Logan, Utah), 100 μg/mL L-glutamine, 50 μg/mL heparin, 30 μg/mL endothelial cell growth supplement (Sigma, St Louis, Mo.), 100 U/mL penicillin, and 100 μg/mL streptomycin (1% pcn/strp, Invitrogen, Carlsbad Calif.). Endothelial cells were plated at a density of 25,000 cells/cm² in 24 well plates and allowed to grow to confluence over a period of 2-3 days.

### Microarray Analysis

Triplicate RNA samples from each of the brain and non-brain derived cell lines were extracted using a Qiagen RNeasy kit (Qiagen, Valencia Calif.). RNA sample quality was verified using an Agilent Bioanalyzer. The samples were processed by the Duke Microarray Facility, which amplified the RNA samples one round (Ambion AmpII, Ambion, Austin, Tex.), labeled the samples with Cy5 dye, and then hybridized the samples to the Operon Human version 4 oligonucleotide array (Operon, Huntsville, Ala.).

### Isolation of Murine Bone Marrow HSCs

All animal procedures were performed in accordance with a Duke University IACUC approved animal use protocol. Stem-cell enriched hematopoietic cells were isolated from the bone marrow of C57/BL6 female mice and congenic B6.SJL-Ptprca Pep3b/BoyJ (B6.SJL) mice (Jackson Laboratory, Bar Harbor, Me.) femurs as follows. The femurs were dissected and the bone marrow was flushed out with cold PBS (Invitrogen) supplemented with 10% FBS and 100 U/mL penicillin, and 100 μg/mL streptomycin. The flushed marrow was strained of debris in a 70 um cell strainer and red blood cells were lysed in red cell lysis buffer (Sigma Aldrich). The lineage committed cells were removed using a lineage depletion column (Miltenyi Biotec Inc, Auburn Calif.).

Multiparameter flow cytometry was conducted to isolate purified HSC subsets. Lin- cells were stained with fluoroscein isothiocyanate (FITC)-conjugated anti-CD34 (eBioscience, San Diego, Calif.), phycoerythrin (PE)-conjugated anti-sca-1, and allophycocyanin (APC)-conjugated anti-c-kit antibodies (Becton Dickinson[BD], San Jose, Calif.), or the appropriate isotype controls. Sterile cell sorting was conducted on a BD FACSVantage SE flow cytometer, using FACSdiva software (BD). Dead cells stained with 7-aminoactinomycin D (7-AAD; BD) were excluded from analysis and sorting. Purified CD34-c-kit+sca-1⁺lin- (34⁻KSL) or KSL subsets were collecting into Iscove's Modified Dulbecco's Medium (IMDM)+10% FBS+1% pcn/strp.

### Co-Culture Studies

Co-culture experiments with endothelial cells were conducted in non-contact conditions using 0.40 □m transwell inserts (Corning, Lowell Mass.). Endothelial growth medium was aspirated and the endothelial monolayer was rinsed twice with PBS prior to insertion of the transwell. Co-culture studies were conducted in HSC cytokine medium (TSF).

### Congenic Competitive Repopulation Units Assay

34⁻KSL cells from B6.SJL mice, carrying the CD45.1 allele, were sorted into 96-well U-bottomplates (BD) containing IMDM+10% FBS+1% pcn/strp. Day 0 34⁻KSL cells were either isolated for injection into recipient animals, or placed into cultures containing TSF, TSF+recombinant PTN, co-culture with HUBECs+goat IgG, or HUBECs+goat anti-PTN. Recipient C57BL6 animals, expressing the CD45.2 allele, received an LD100/30 dose of 950 cGy total body irradiation (TBI) using a Cs 137 irradiator and then transplanted via tail vein injection with 10, 30 or 100 34⁻KSL cells or their progeny following culture. A rescue dose of 1×10⁵ non-irradiated CD45.2 MNCs were co-injected into recipient mice. Multi-lineage hematologic reconstitution was monitored in the peripheral blood (PB) by flow cytometry, as previously described, at 4, 8, 12, and 24 weeks posttransplant. PB was collected via submandibular puncture; cells were treated with RBC lysis buffer (Sigma-Aldrich), and washed twice prior to staining with FITC- or PE-CD45.1, FITC-CD45.2, PE-anti-Thy 1.2, APC-anti-B220, APC-anti-Ter-119, or PE-anti-Mac-1 and PE-anti-Gr1. Animals were considered to be engrafted if donor CD45.1 cells were present at >1% for all lineages (Zhang et al, Nat. Med. 12:240-245 (2006)).

Radioprotective cell frequency and Competitive Repopulating Unit (CRU) calculations were performed using L-Calc software (Stem Cell Technologies) (Zhang et al, Nat. Med. 12:240-245 (2006), Bonnefoix et al, J. Immunol. Methods 194:113-11.9 (1996)).

### Direct ELISA

Triplicate samples of conditioned medium from the HUBEC line used for the co-culture studies were incubated overnight in an 96-well ELISA plate along with standard amounts of human recombinant PTN. The ELISA specific reagents were purchased from R&D systems. The plates were rinsed, blocked for 1 hour with 3.5% Bovine Serum Albumin (BSA) in PBS, incubated for 1 hr with biotinylated 1 ug/ml anti-PTN, rinsed, incubated for 30 minutes with HRP conjugated streptavidin. The plates were developed with Color Substrate Solution followed by Stop Solution and the fluorescence was measured on a plate reader.

### Results

### Pleiotrophin is Secreted by HUBECs and Accounts for the Amplification of HSCs Observed in HUBEC Culture

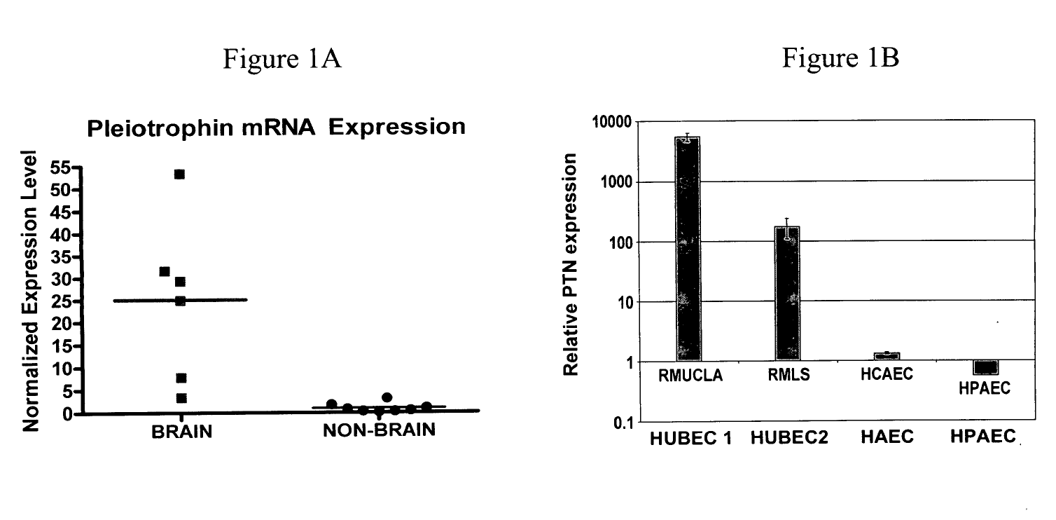

Co-culture of human and murine HSCs with HUBECs in non-contact culture induces a 1-log expansion of long-term repopulating HSCs in short term (7 day) culture. In gene expression analysis and via RTPCR, it was found that PTN is markedly overexpressed by 10-33 fold in HUBECs as compared to non-brain EC lines (FIG. 1). Experiments were carried out to test whether PTN is required for the effect of HUBEC co-culture on HSC expansion to occur. For these studies, a blocking anti-PTN antibody (R&D Systems, Minneapolis, Minn.) was used which was added to cultures in which 1-10×10³ CD34-c-kit+sca-1⁺lin- (34⁻KSL) cells were cultured in non-contact conditions with HUBECs (C57Bl6 bone marrow (BM) 34⁻KSL cells were used). 34⁻KSL cells have been previously shown to be highly enriched for HSC content to the level of 1 per 10-100 cells and these cells can be isolated via antibody staining and flow cytometric sorting (Chute et al, Stem Cells 22:202-215 (2004), Chute et al, Blood 105:576-583 (2005), Chute et al, Blood 100:4433-4439 (2002)). The HUBEC co-cultures were also supplemented with Iscove's Modified Dulbecco's Medium (IMDM) supplemented with thrombopoietin 50 ng/mL, SCF 120 ng/mL and Flt-3 ligand 20 ng/mL as previously described (Chute et al, Stem Cells 22:202-215 (2004), Chute et al, Blood 105:576-583 (2005), Chute et al, Blood 100:4433-4439 (2002)). It was observed that HUBEC co-cultures supplemented, only with isotype IgG antibody supported a significant expansion of KSL stem/progenitor cells compared to cytokines alone. The HUBEC plus anti-PTN group also demonstrated a significant increase in KSL cells compared to cytokines alone. Analysis of colony forming cell (CFC) content, which is a measure of committed progenitor cells rather than HSCs, demonstrated that HUBEC plus anti-PTN cultures contained significantly less CFCs compared to HUBECs supplemented with isotype alone.

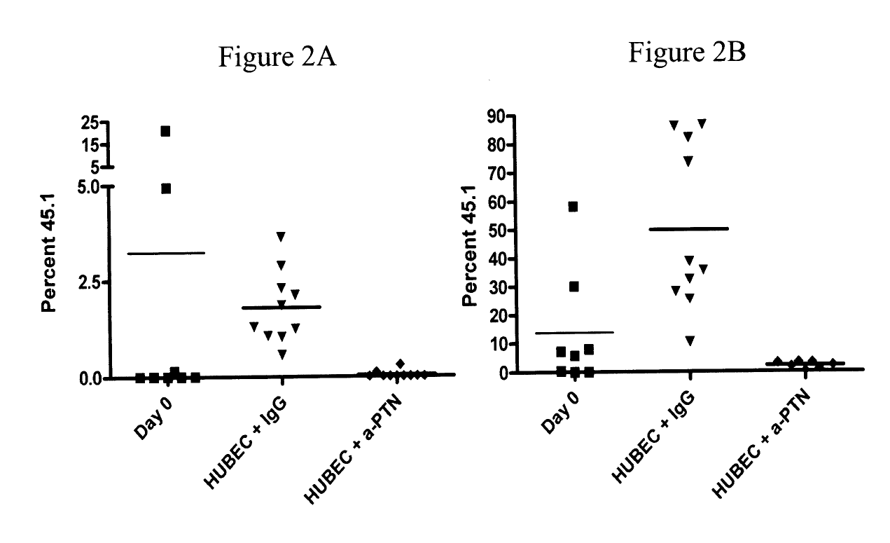

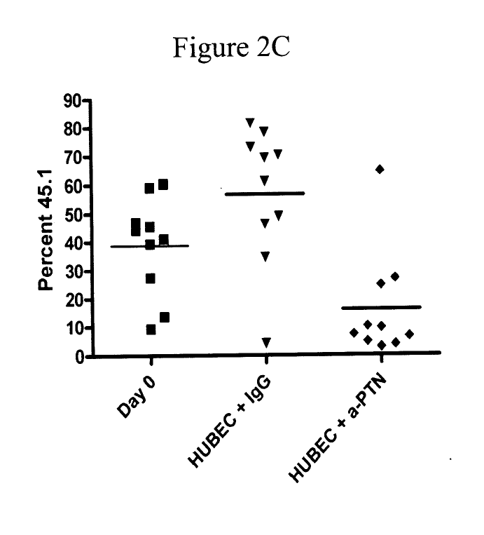

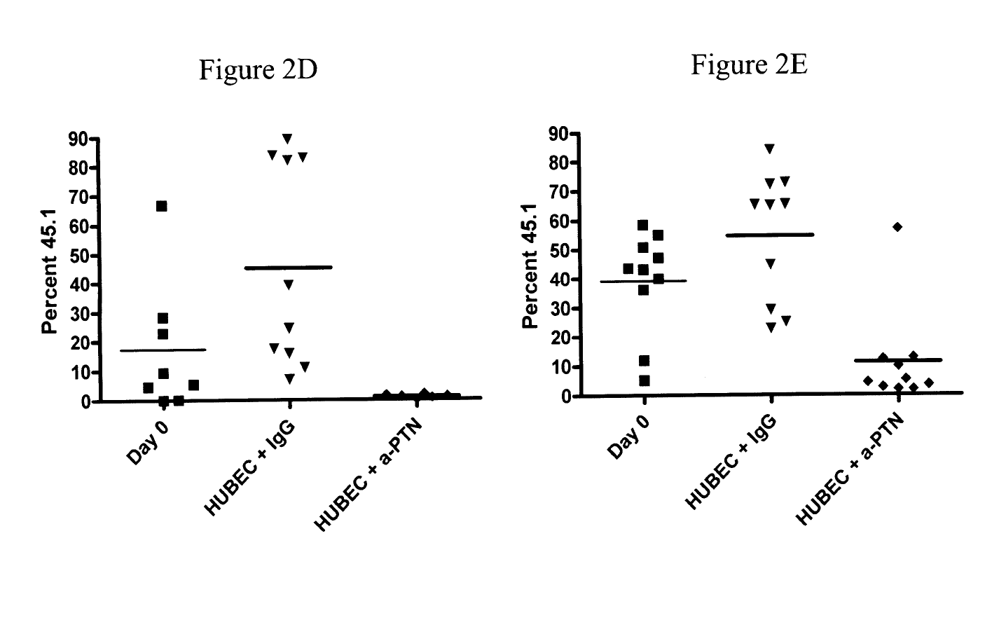

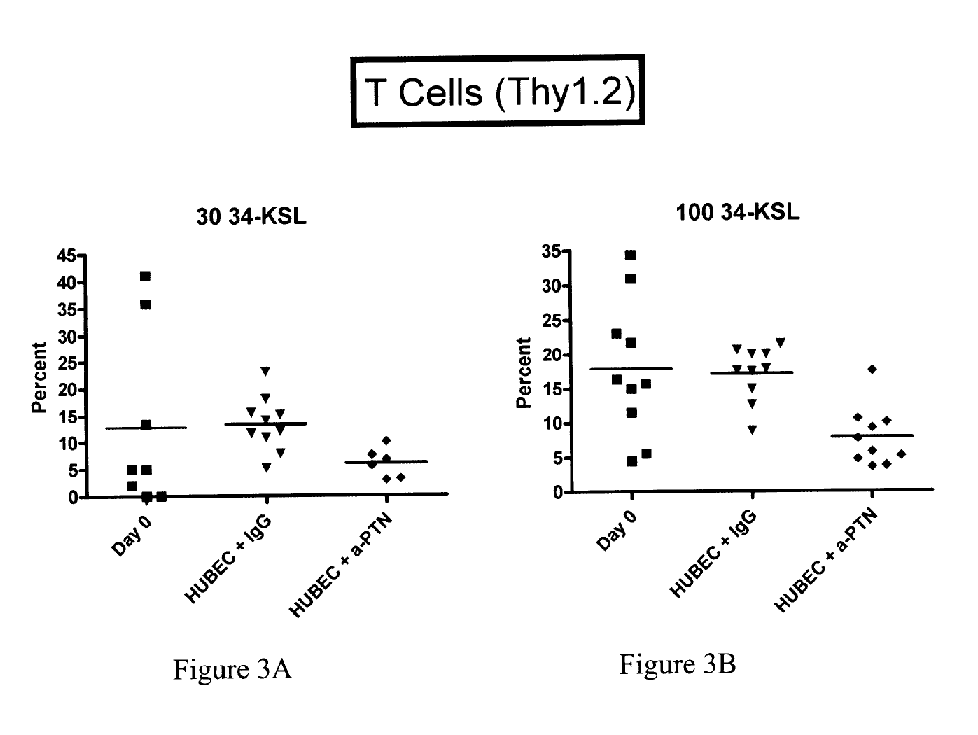

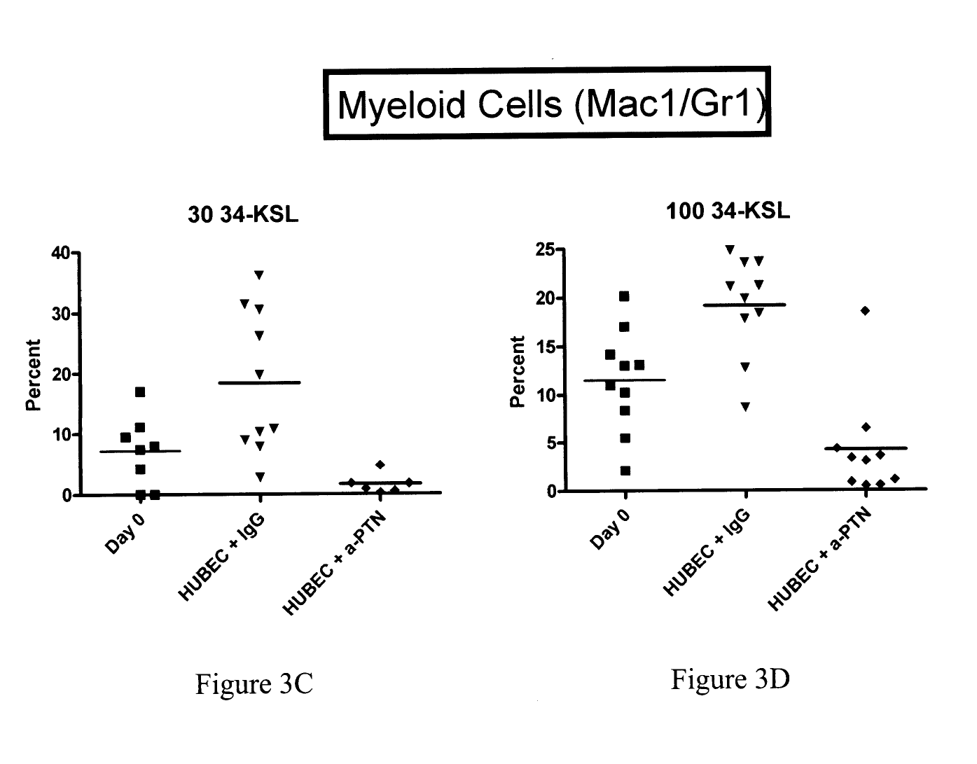

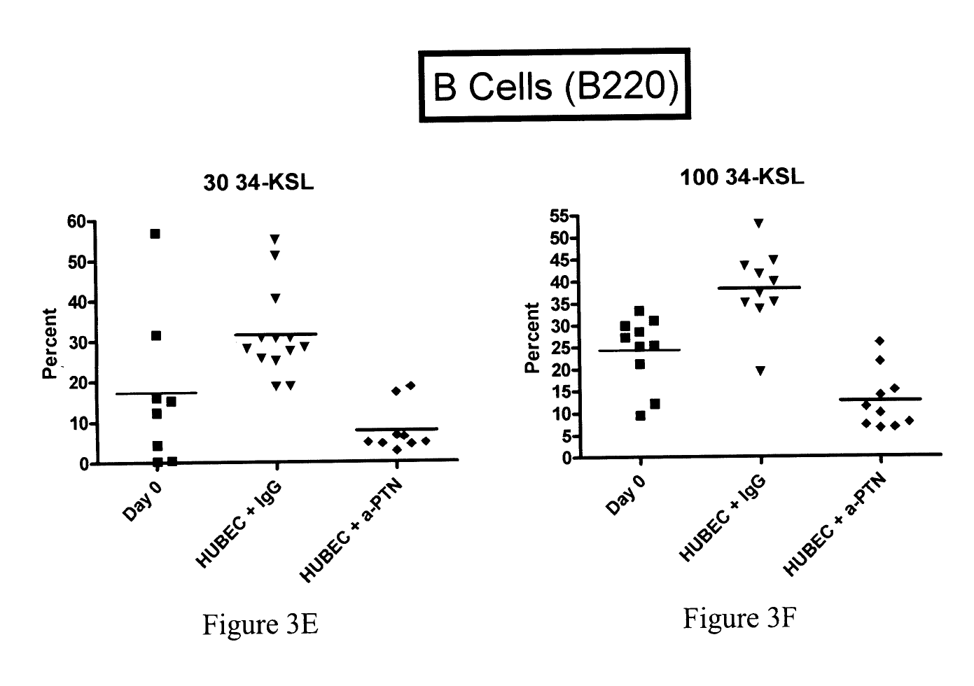

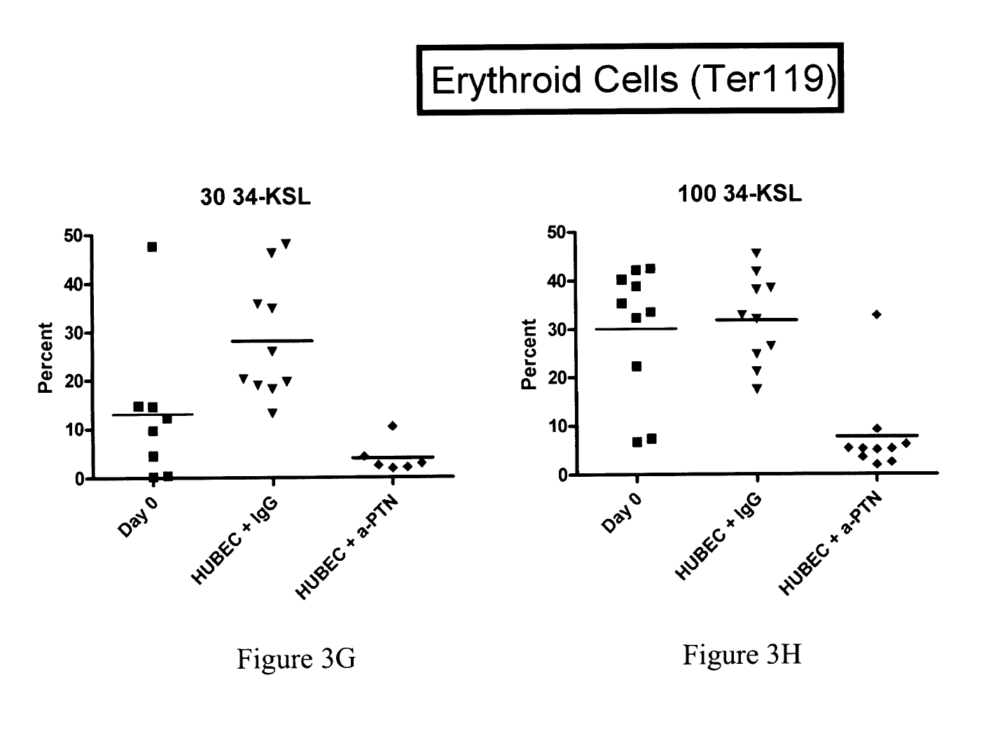

A determination was next made as to whether the addition of anti-PTN to HUBEC cultures could alter the estimate of HSC content within these cultured progeny compared to input 34⁻KSL cells and the progeny of cytokines alone vs. HUBEC plus isotype antibody. HUBEC co-cultures supported an 8 fold increase in long-term repopulating HSCs compared to input 34⁻KSL cells and cytokine treated progeny (FIG. 2). Remarkably, the progeny of HUBECs plus anti-PTN demonstrated essentially a complete loss of HSC content, suggesting that blockade of PTN signaling prevented the amplification of HSCs in culture that was otherwise mediated by HUBECs. Multilineage analysis also demonstrated that mice transplanted with HUBEC cultured progeny displayed increased myeloid, B cell and erythroid progenitor cell contribution compared to day 0 34⁻KSL cell transplants or the progeny of TSF alone. Interestingly, mice transplanted with the progeny of HUBECs plus anti-PTN displayed a nearly complete loss of donor-derived myeloid, B cell and erythroid progenitor cell production compared to the other groups (FIG. 3). Importantly, the elimination of HSC activity within the HUBEC-cultured progeny via treatment with anti-PTN was observed at the 4 week, 8 week and 12 week analysis points, demonstrating that both short-term HSCs and long-term HSCs were affected by blockade of PTN signaling. Taken together, these data demonstrate that PTN is produced by HUBECs and is a critical growth factor for HSCs and triggers the self-renewal of HSCs in vitro.

### The Addition of PTN Expands HSCs in Liquid Suspension Culture

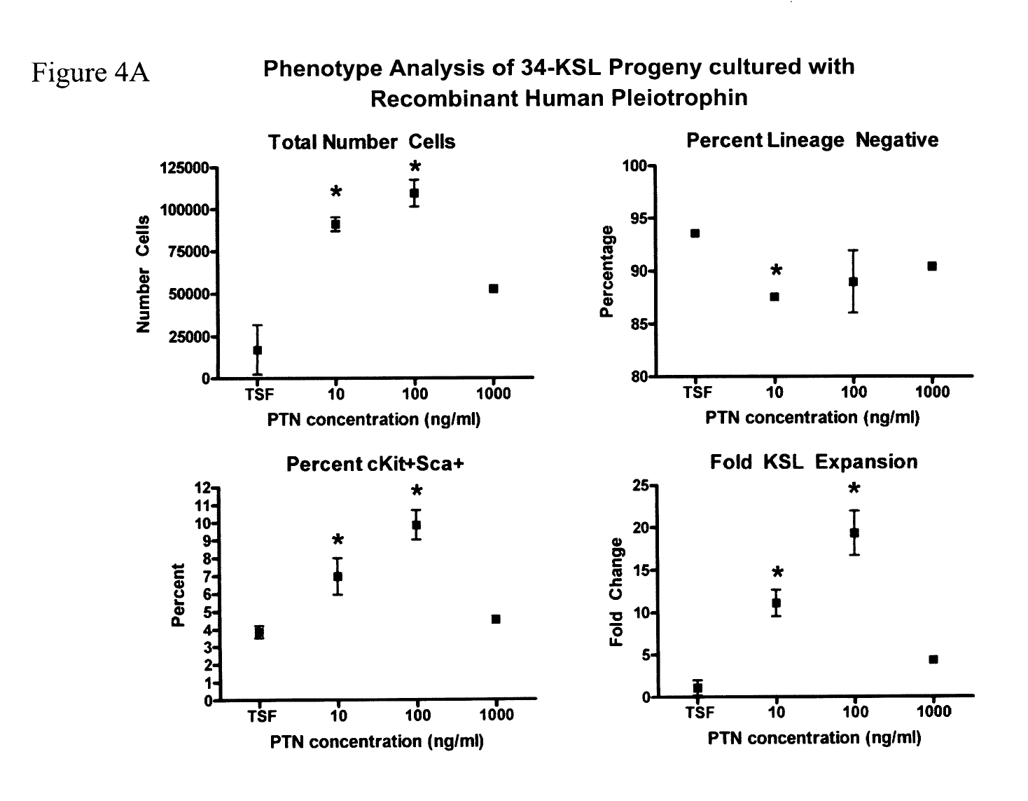

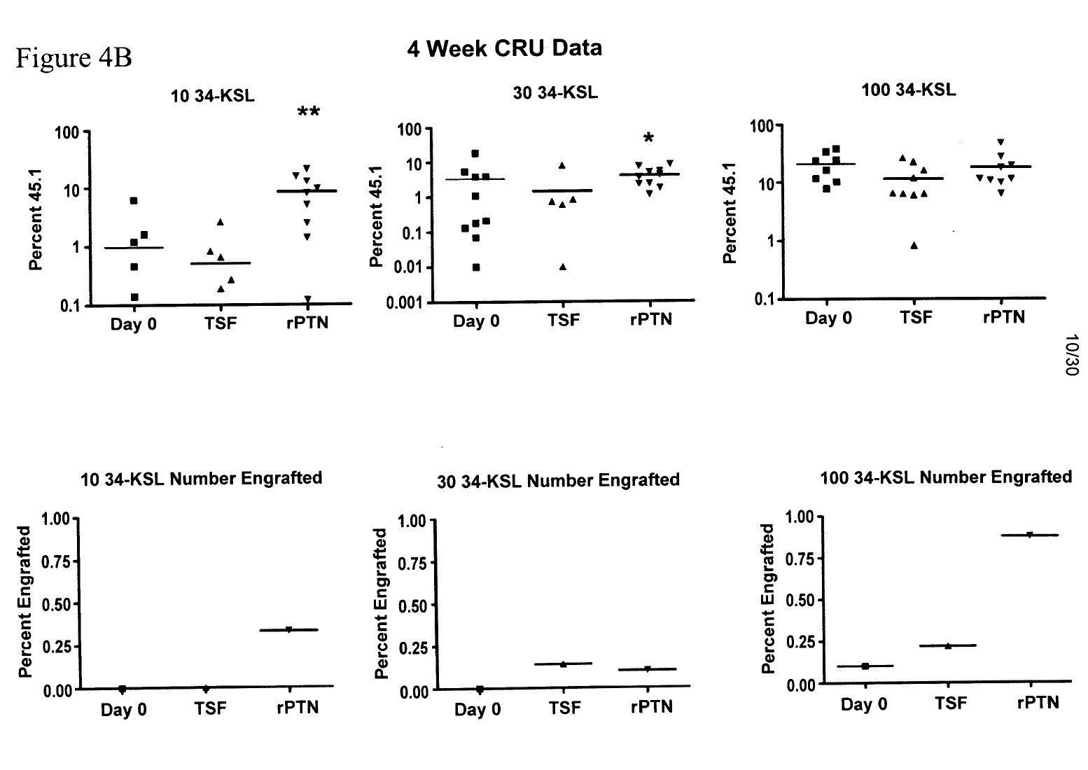

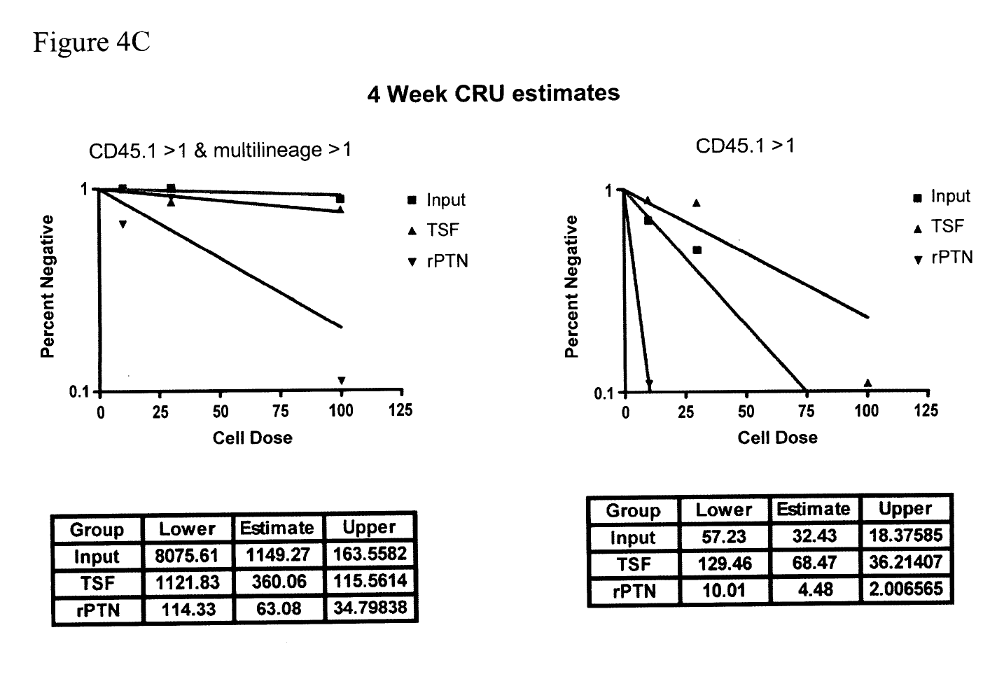

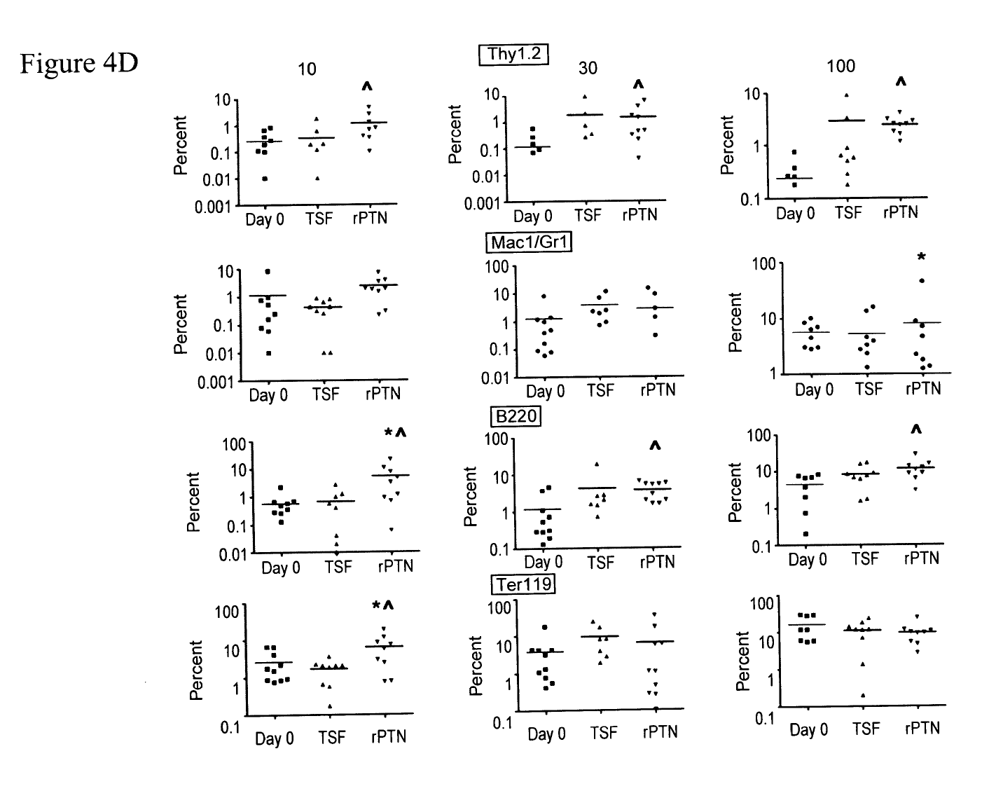

The above “loss of function” studies strongly implicated PTN as a secreted growth factor for HSCs. In order to prove that PTN alone stimulated the proliferation of HSCs in culture, outside the context of a supportive microenvironment, murine 34⁻KSL cells were placed in liquid suspension culture with thrombopoietin 50 ng/mL, SCF 120 ng/mL and flt-3 ligand 20 ng/mL (TSF) with and without increasing concentrations (10, 100 and 1000 ng/mL) of recombinant PTN (rPTN) (R & D Systems, Minneapolis, Minn.) and compared total cell expansion, phenotypic changes and HSC functional assays. The addition of increasing doses of PTN caused a significant increase in total cells (P<0.001) and KSL cells in culture (P<0.001) compared to the progeny of cytokines alone and a dose response effect was observed (FIG. 4A). These data suggested that PTN was indeed a growth factor for HSCs but in order to prove this, competitive repopulating assays were performed as described below.

For the competitive repopulating assays, recipient CD45. 2⁺ mice were lethally irradiated with 950 cGy TBI and subsequently transplanted via tail vein with limiting doses (10, 30 or 100 cells) of donor CD45. 1⁺34⁻KSL cells or their progeny following culture with TSF alone or TSF plus PTN (100 ng/mL). Host BM cells (1×10⁷) were co-transplanted as competitor cells. At 4 weeks following transplantation, mice transplanted with day 0 34⁻KSL cells showed no CD45. 1⁺ donor derived multilineage engraftment at the 10 or 30 cell dose and only low level engraftment at the 100 cell dose (FIG. 4B). Similarly, the progeny of 34⁻KSL cells cultured with TSF alone also showed little or no multilineage CD45. 1⁺ donor cell derived engraftment at 4 weeks. Conversely, the progeny of the same doses of 34⁻KSL cells cultured with TSF plus PTN demonstrated donor derived multilineage engraftment in up to 50% of mice transplanted at 4 weeks, indicating that an expansion of short-term HSCs had occurred in culture in response to PTN treatment. Poisson statistical analysis demonstrated that the day 0 34⁻KSL cells contained a frequency of 1 in 32 HSCs (95% Confidence Interval (CI): 18-57), whereas the progeny of 34⁻KSL cells cultured with TSF had an HSC frequency of 1 in 69 cells (CI:36-130). In contrast, the HSC frequency within the progeny of 34⁻KSL cells cultured with TSF plus PTN was 1 in 4 cells (CI:2-10) (FIG. 4C). These results confirmed that PTN is a growth and self-renewal factor for HSCs and longer term analyses of the transplanted mice will verify whether long-term repopulating HSCs were expanded significantly in response to PTN treatment.

Lastly, in order to determine whether PTN treatment caused a skewing or lineage restriction of HSCs following transplantation in vivo, the lineage repopulation of erythroid, myeloid and lymphoid cells in vivo was examined in transplanted mice. As shown in FIG. 4D, mice transplanted with the progeny of 34⁻KSL cells treated with TSF plus PTN demonstrated multilineage engraftment of myeloid, erythroid, B lymphoid and T lymphoid progeny which was comparable in distribution to the progeny of unmanipulated 34⁻KSL cells following transplantation. These results confirmed that treatment of HSCs with PTN did not alter the normal multilineage differentiation potential of HSCs.

### Example 2

### Experimental Details

### EC Cultures and Microarray Analysis

Primary human EC lines derived from uterine, umbilical, iliac, dermal, coronary and pulmonary arteries (Lonza, Gaithersburg, Md.) were cultured according to manufacturer's guidelines. Primary HUBECs were generated and cultured in complete EC culture media as previously described (Chute et al, Blood 100:4433-9 (2002), Chute et al, Blood 105: 576-83 (2005)). RNA from n=6 HUBECs and n=8 non-brain ECs were amplified and hybridized to a human oligonucleotide spotted microarray (Operon, Huntsville, Ala.). The microarray data were analyzed using an unsupervised hierarchical cluster analysis and the gene list was screened for annotated soluble proteins. Sample processing and hybridization to Operon Human Arrays (Operon) were performed as previously described (Dressman et al, PLoS Medicine 4:690-701 (2007)).

### Isolation of BM HSCs and In Vitro Cultures

Purified BM 34-KSL cells were isolated from C57Bl6 and B6.SJL mice (Jackson Laboratory, Bar Harbor, Me.) via flow cytometric cell sorting as previously described (Reya, et al, Nature 423:409-14 (2003), Salter et al, Blood 113:2104-7 (2009)). Liquid suspension cultures of BM 34⁻KSL cells were supplemented with IMDM+10% FBS+1% pcn/strp+20 ng/ml thrombopoietin, 120 ng/ml SCF, and 50 ng/ml flt-3 ligand (“TSF” media) with and without recombinant (human) PTN (R&D Systems, Minneapolis, Minn.). Non-contact HUBEC cultures were conducted using 0.4 μm transwell inserts (Corning, Lowell Mass.) and supplemented with TSF media with and without goat anti-PTN or isotype control antibody (R&D). Phenotypic analysis for KSL cells was performed as previously described (Chute et al, Blood 109:2365-72 (2007), Salter et al, Blood 113:2104-7 (2009)).

### CRU Assays

BM 34⁻KSL cells were either isolated for injection into recipient animals, or placed into cultures containing TSF alone, TSF+PTN, TSF+HUBECs+goat IgG, or TSF+HUBECs+goat anti-PTN. Recipient C57BL6 animals (CD45. 2⁺) received 950 cGy total body irradiation (TBI) and were then injected via tail vein with limiting doses of BM 34⁻KSL cells or their progeny following culture. 1×10⁵ host BM MNCs were co-injected into recipient mice as competitor cells. Multilineage hematologic reconstitution was measured in the PB by flow cytometry over time post-transplant as previously described (Reya, et al, Nature 423:409-14 (2003), Salter et al, Blood-113:2104-7 (2009)). Animals were considered to be engrafted if donor CD45. 1⁺ cells were present at ≧1% in the PB (Chute et al, Blood 100:4433-9 (2002), Chute et al, Blood 105: 576-83 (2005), (Chute et al, Proc Natl Acad Sci USA 103, 11707-12 (2006)). CRU estimates were performed using L-Calc software (Stem Cell Technologies) as previously described (Reya, et al, Nature 423:409-14 (2003), Chute et al, Blood 109:2365-72 (2007), Chute et al, Proc Natl Acad Sci USA 103, 11707-12 (2006)).

Secondary competitive transplant assays were performed using whole BM harvested from primary CD45. 2⁺ mice at 24 weeks following transplantation with either CD45. 1⁺ BM 34⁻KSL cells or the progeny of 34⁻KSL cells following culture with TSF alone or TSF+PTN. Secondary recipient CD45. 2⁺ C57Bl6 mice were irradiated with 950 cGy TBI and PB analysis of donor cell engraftment was performed at 12 weeks post-transplantation in secondary mice.

### Quantitative RT-PCR and Direct ELISA

RT-PCR analyses of PTN in ECs and HES-1, GFI-1 and PTEN in BM KSL cells and FACS-sorted KSL cells following culture were performed using a 2-step RTPCR reaction as previously described (Chute et al, Proc Natl Acad Sci USA 103, 11707-12 (2006)). Conditioned medium (CM) was generated from HUBECs and non-brain ECs as previously described (Chute et al, Blood 100:4433-9 (2002), Chute et al, Blood 105: 576-83 (2005)) and ELISA for PTN was performed following manufacturer's guidelines.

PI 3-kinase and β-catenin assays

For analysis of RPTPβ/ζ in hematopoietic cells, cytospins of BM MNCs were generated (˜10,000 cells/slide). Rat anti-RPTPβ/ζ (BD) or rat IgG was added and a FITC anti-rat secondary antibody was utilized. Flow cytometric analysis was performed on BM KSL cells to confirm RPTPβ/ζ expression. Wortmannin (Cell Signaling Technology, Danvers, Mass.) was added to HSC cultures at 1 μM to inhibit PI3 kinase activity. For analysis of pAkt, BM KSL cells were incubated overnight with a primary antibody to Akt phosphorylated at S473, following manufacturer's guidelines (BD). Transgenic β-catenin^{-/-} (loxP,loxP;Vav-cre) mice were a gift from T. Reya, Duke University. Immunofluorescence analysis for the activated β-catenin was performed using cytospins of BM KSL cells or their progeny and staining with antibody against non-phosphorylated β-catenin (Clone 8E7, Upstate Biotechnology, Lake Placid, N.Y.) or isotype control, and goat antimouse alexa-fluor 488 (BD) (Congdon et al, Stem Cells 26:1202-10 (2008)).

### In Vivo PTN Studies

Adult B6.SJL mice received a single fraction of 700 cGy TBI and were then treated either with PBS (saline) or 2 μg PTN intraperitoneally daily×7 days (beginning 4 hours post irradiation). At day +7, the mice were sacrificed and total viable BM cells were quantified. Flow cytometric analysis was performed to estimate the percentage of BM KSL cells in each femur (Chute et al, Blood 109:2365-72 (2007), Salter et al, Blood 113:2104-7 (2009)). Colony forming cell (CFC) assays were performed using MethoCult M3434 media (Stem Cell Technologies, Vancouver, BC) as previously described (Chute et al, Blood 109:2365-72 (2007), Salter et al, Blood 113:2104-7 (2009)). Long-term cultureinitiating cell (LTC-IC) assays were performed as follows: Murine M2-10B4 (ATCC CRL-1972) BM stromal cells were plated in a 24 well dish and irradiated with 1500 cGy. Limiting dilutions (45,000, 90,000, and 180,000) of BM MNCs from irradiated mice that were treated with either PTN or PBS were added to the stromal cell layers and maintained in MyeloCult M5300 media (Stem Cell Technologies) with weekly half-medium changes for 4 weeks. At 4 weeks, the non-adherent and adherent cells (15,000 cells/dish) were collected and plated into 3×35 mm dishes (MethoCult, StemCell Technologies). After two weeks, hematopoietic colonies were counted and scored.

### Results

Treatment with PTN Induces the Expansion of Phenotypic HSCs

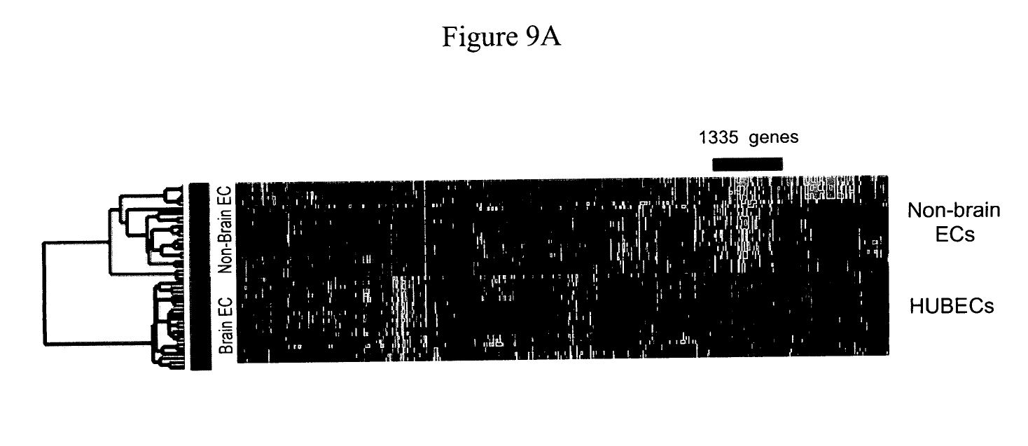

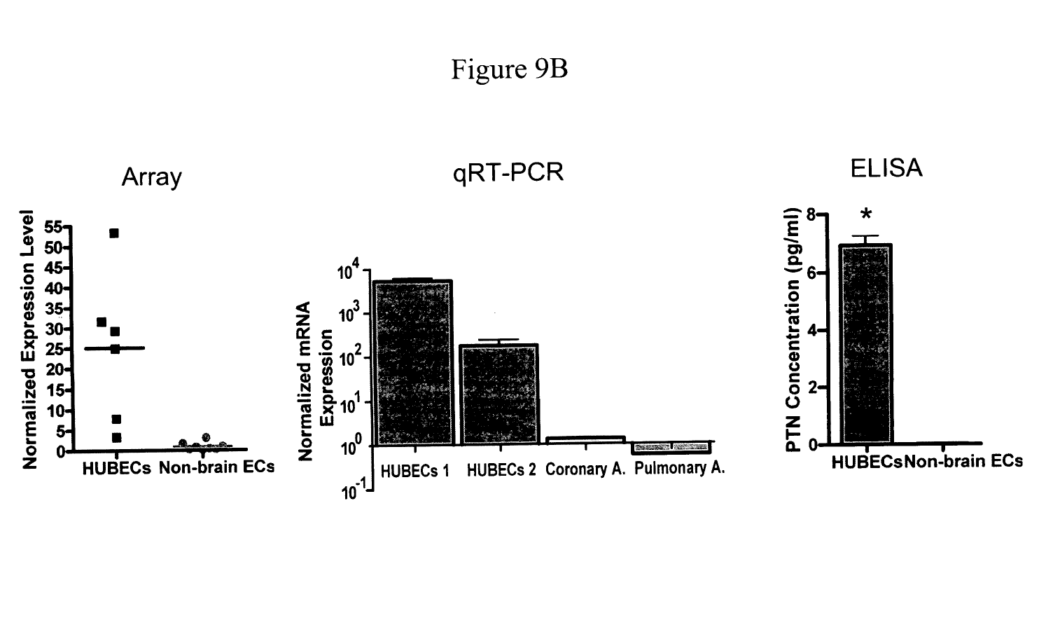

It has been shown previously that adult sources of human endothelial cells (ECs) support the expansion of human HSCs in short-term culture (Chute et al, Blood 105: 576-83 (2005), Chute et al, Blood 109:2365-72 (2007)). In contrast to co-culture studies with stromal cells (Gottschling et al, Stem Cells 25:798-806 (2007)), which have demonstrated a requirement for cell-to-cell contact for HSC maintenance in vitro, it has been shown that primary human brain endothelial cells (HUBECs) produce a soluble activity capable of inducing a 1-log expansion of human HSCs ex vivo (Chute et al, Blood 100:4433-9 (2002), Chute et al, Blood 105: 576-83 (2005)). In order to identify the HUBEC-secreted proteins responsible for this HSC-amplifying activity, genome-wide expression analysis of HUBECs was performed as compared to nonbrain human ECs which lack HSC-supportive activity (FIG. 9A). Thirteen genes were identified that were >5-fold overexpressed in HUBECs and produced soluble gene products (Table 1). It was found that the expression of PTN, a heparin-binding growth factor with no known role in hematopoiesis (Meng et al, Proc Natl Acad Sci USA 97: 2603-8 (2000)), was 25-fold higher in HUBECs versus non-brain ECs (FIG. 9B). Quantitative RT-PCR confirmed a >100-fold increase in the expression of PTN in HUBECs versus non-brain ECs and ELISA of HUBEC-conditioned media (1×) demonstrated an increased concentration of PTN of 6.9±0.3 pg/ml compared to 0.02±0.01 pg/mL in non-brain ECconditioned media (P=0.04, FIG. 9B).

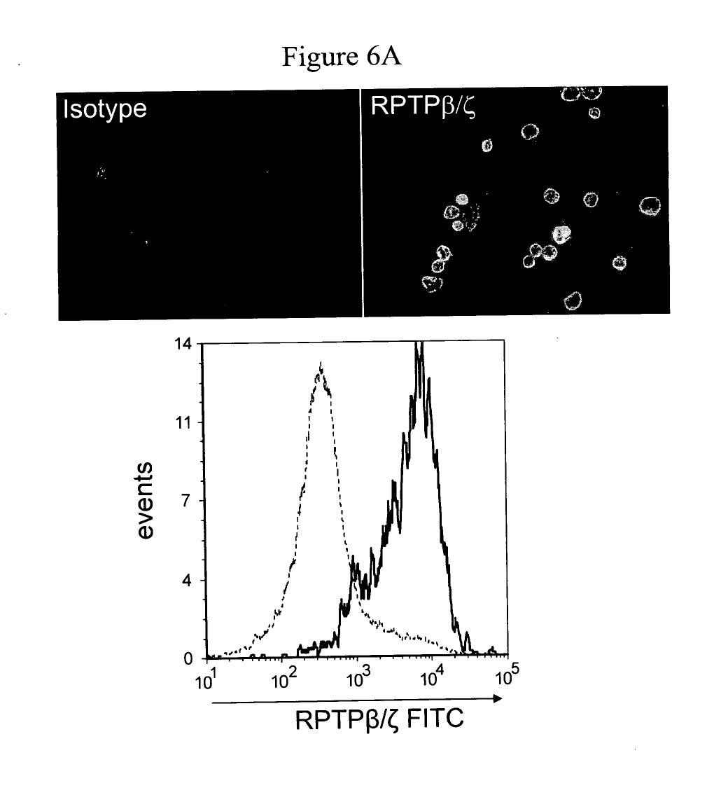

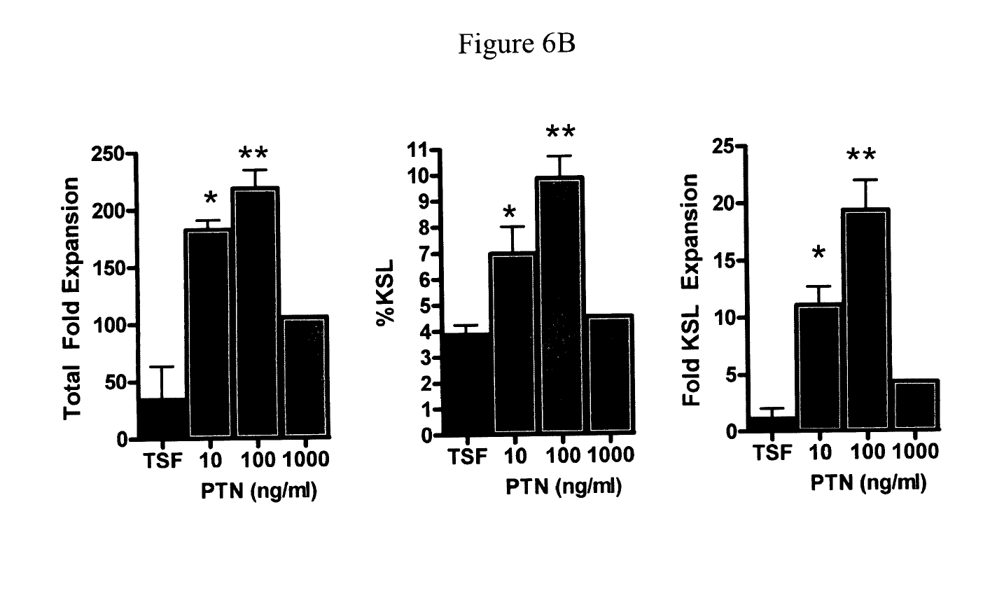

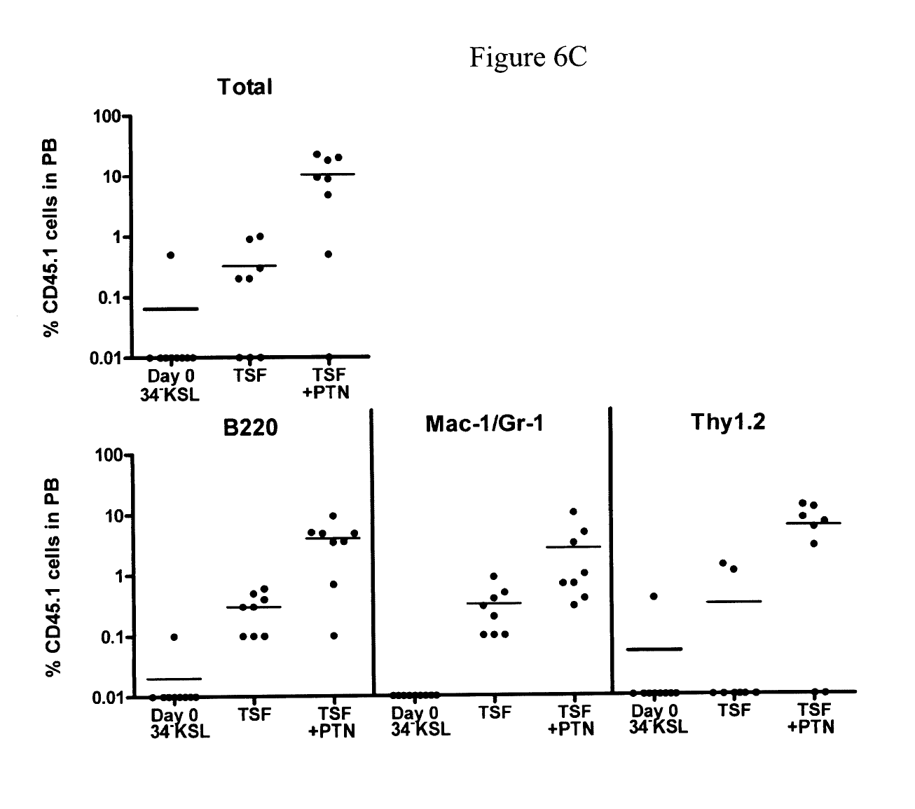

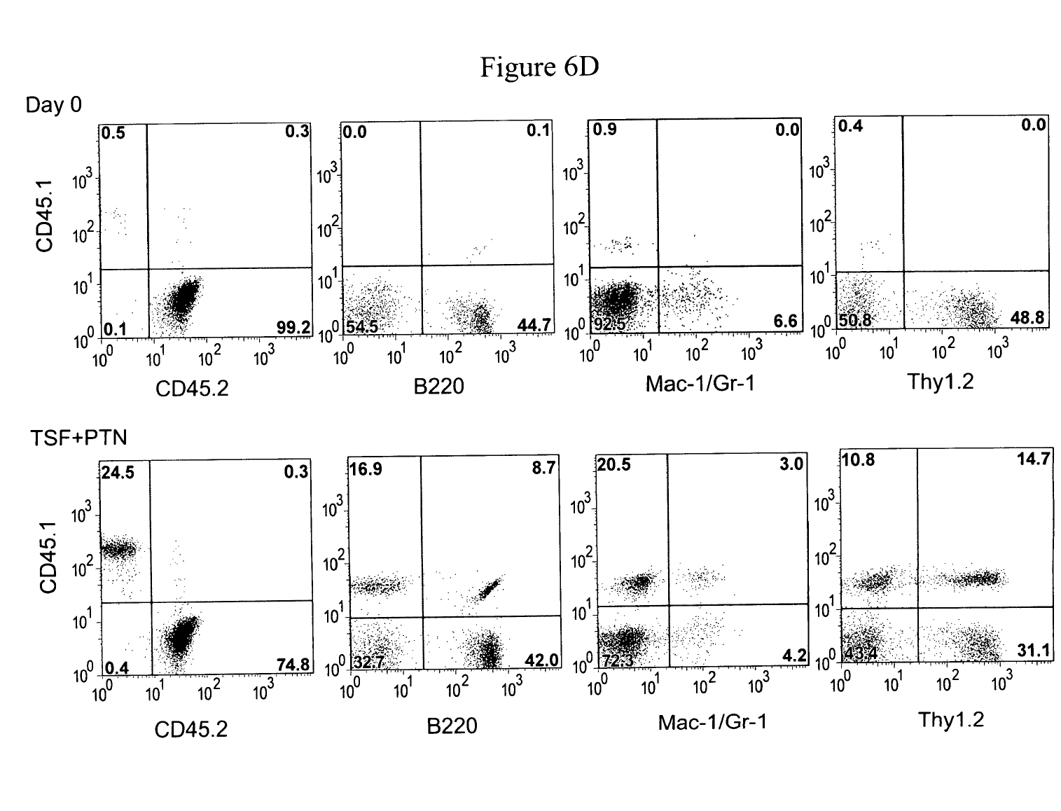

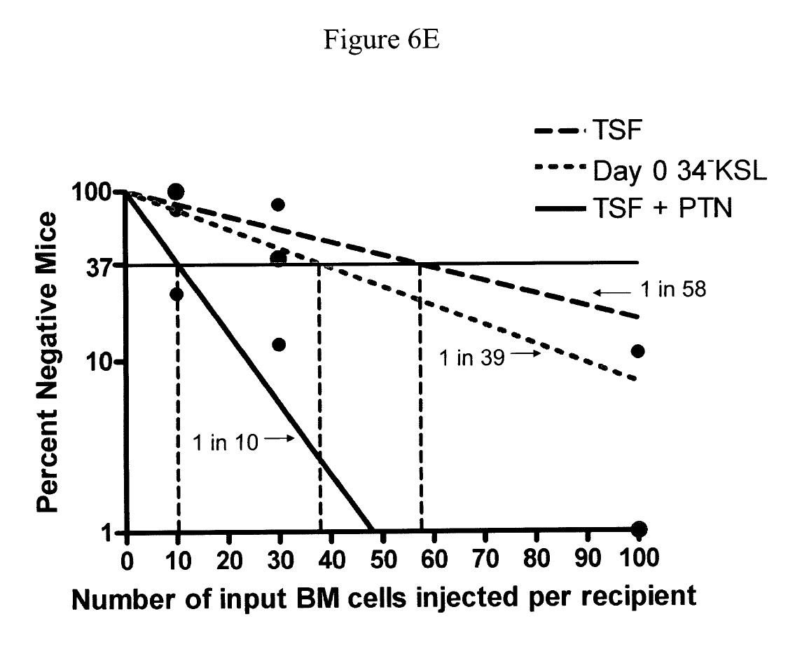

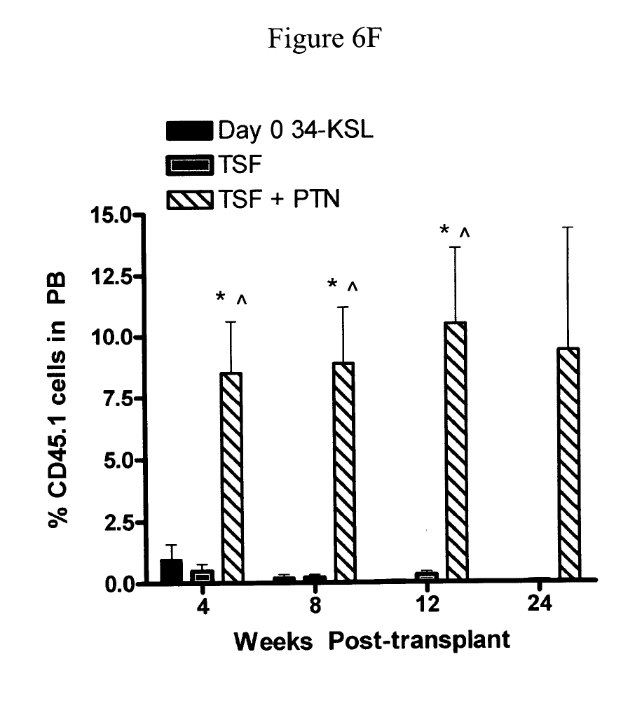

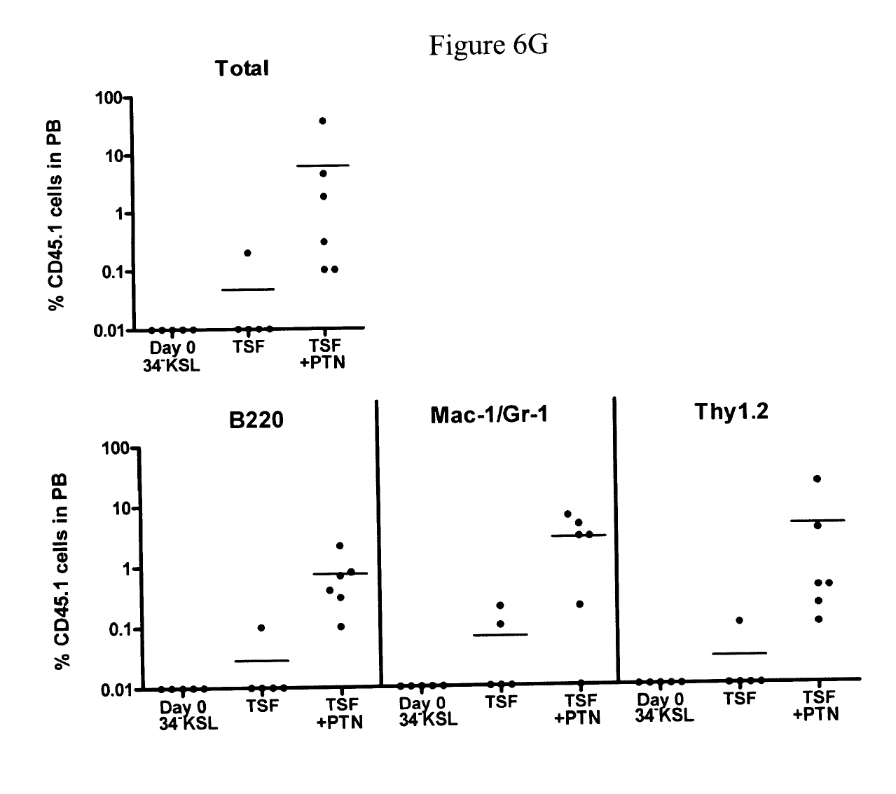

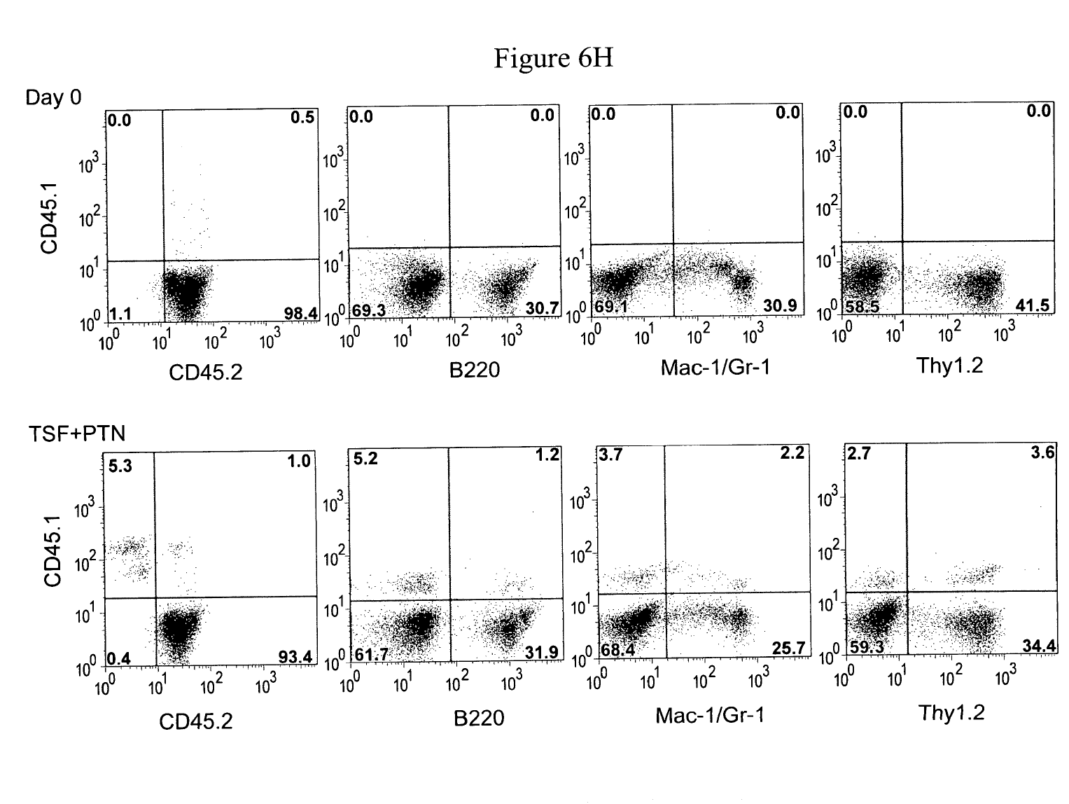

Since PTN has no known function in regulating hematopoiesis (Meng et al, Proc Natl Acad Sci USA 97: 2603-8 (2000)), an examination was first made as to whether BM stem/progenitor cells expressed one or more of the PTN receptors, receptor protein tyrosine phosphatase β/ζ (RPTP β/ζ) Syndecan or anaplastic lymphoma kinase (ALK) (Stoica et al, J Biol Chem 276:16772-9 (2001), Landgraf et al, J Biol Chem 283:25036-45 (2008)). The majority of BM MNCs and c-kit+sca-1⁺lin⁻ (KSL) stem/progenitor cells expressed RPTP β/ζ (n=3, mean 87.0%±8.8 and 89%, respectively; FIG. 6A), whereas neither population expressed Syndecan or ALK (data not shown). In order to determine whether PTN might be a growth factor for HSCs, C57Bl6 BM CD34-KSL cells, which are highly enriched for HSCs (Kiel et al, Nat Rev Immunol 8:290-301 (2008), Salter et al, Blood 113:2104-7 (2009)), were isolated by FACS and placed in liquid suspension culture with 20 ng/mL thrombopoietin, 120 ng/mL SCF and 50 ng/mL Flt-3 ligand (“TSF”) with or without 10, 100 or 1000 ng/mL PTN. A dose responsive increase was observed in total cells, % KSL cells and total KSL cells in response to the addition of 10 to 100 ng/mL of PTN (FIG. 6B). The addition of 100 ng/mL PTN to TSF caused a 6.4-fold increase in total cells and a 17.7-fold increase in total KSL stem/progenitor cells compared to TSF alone (P=0.005 and P=0.006, respectively, FIG. 6B).

Treatment with PTN is Sufficient to Induce the Expansion of LT-HSCs

In order to determine if treatment with PTN could induce functional HSC expansion in culture, competitive repopulating unit (CRU) assays were performed using limiting dilutions of donor CD45. 1⁺ BM 34⁻KSL cells transplanted into lethally irradiated CD45. 2⁺ C57Bl6 mice. Peripheral blood (PB) was collected from primary recipient mice at 4 weeks, 12 weeks and 24 weeks to assess the engraftment of donor CD45. 1⁺ cells in the PB of recipient mice. At 12 weeks post-transplant, mice that were transplanted with the progeny of 34⁻KSL cells cultured with TSF+100 ng/mL PTN demonstrated a >10-fold increase in CD45. 1⁺ donor cell engraftment in the PB compared to mice transplanted with the identical dose of day 0 34⁻KSL cells and mice transplanted with the progeny of 34⁻KSL cells cultured with TSF alone (FIG. 6C, P=0.0008 and P=0.001, respectively). These data suggested that PTN caused an expansion of HSCs in culture. The PB engraftment of multilineage donor CD45. 1⁺-derived myeloid, B-lymphoid and T cell progeny was also significantly increased at 12 weeks in mice transplanted with the progeny of PTN-treated 34⁻KSL cells compared to that observed in mice transplanted with the same dose of day 0 34⁻KSL cells or their progeny following culture with TSF alone (FIGS. 6C and 6D). Poisson statistical analysis of a large number of transplanted mice (n=75) demonstrated that the 12 week CRU frequency within BM 34⁻KSL cells was 1 in 39 cells (95% Confidence Interval [CI]: 1/21 to 1/70, FIG. 6E, Table 2). As expected, the CRU frequency within the progeny of 34⁻KSL cells following culture with cytokines alone (TSF) was reduced to 1 in 58 cells (95% CI: 1/31 to 1/108). Conversely, the CRU frequency within the progeny of 34⁻KSL cells cultured with TSF+PTN was 1 in 10 cells (95% CI: ⅕ to 1/20). Therefore, the addition of PTN induced a 4-fold increase in HSCs compared to input and a 6-fold increase compared to the progeny of TSF alone. Mice transplanted with the progeny of TSF+PTN also displayed higher donor CD45. 1⁺ cell reconstitution at all time points through 24 weeks compared to mice transplanted with day 0 34⁻KSL cells or their progeny following culture with TSF alone (FIG. 6F). This corresponded to an increased CRU frequency in the PTN-treated progeny compared to input 34⁻KSL cells at all time points. At 4 weeks, the frequency of short-term CRU was 6.4-fold higher in the progeny of 34⁻KSL cells cultured with TSF+PTN compared to input 34⁻KSL cells (1 in 5 cells, 95% CI: ½- 1/10 versus 1 in 32 cells, 95% CI: 1/18- 1/57). At 24 weeks post-transplant, the CRU frequency was 4-fold increased in the PTN-treated progeny compared to input 34⁻KSL cells (1 in 13, 95% CI: ⅙- 1/30 versus 1 in 52, 95% CI: 1/25- 1/106).

In order to confirm that PTN caused the amplification of long-term repopulating HSCs with serial repopulating capacity, secondary transplants were performed. Importantly, secondary CD45. 2⁺ mice transplanted with BM harvested at 24 weeks from primary recipients of PTN-treated 34⁻KSL cells demonstrated >10-fold higher CD45. 1⁺ cell engraftment at 12 weeks post-transplant compared to secondary mice transplanted with BM from primary mice in the 34⁻KSL cell group or the TSF alone group (P=0.003 and P=0.02, respectively; FIG. 6G); secondary mice transplanted with BM from primary mice that were transplanted with PTN-treated 34⁻KSL cells also demonstrated normal multilineage differentiation at 12 weeks (FIGS. 6G and 6H). Taken together, these data illustrate that treatment with PTN was sufficient to induce a significant expansion of LT-HSCs in culture and this amplification of LT-HSCs did not alter their multilineage differentiation potential.

### Inhibition of PTN Signaling Blocks EC-Mediated Expansion of HSCs

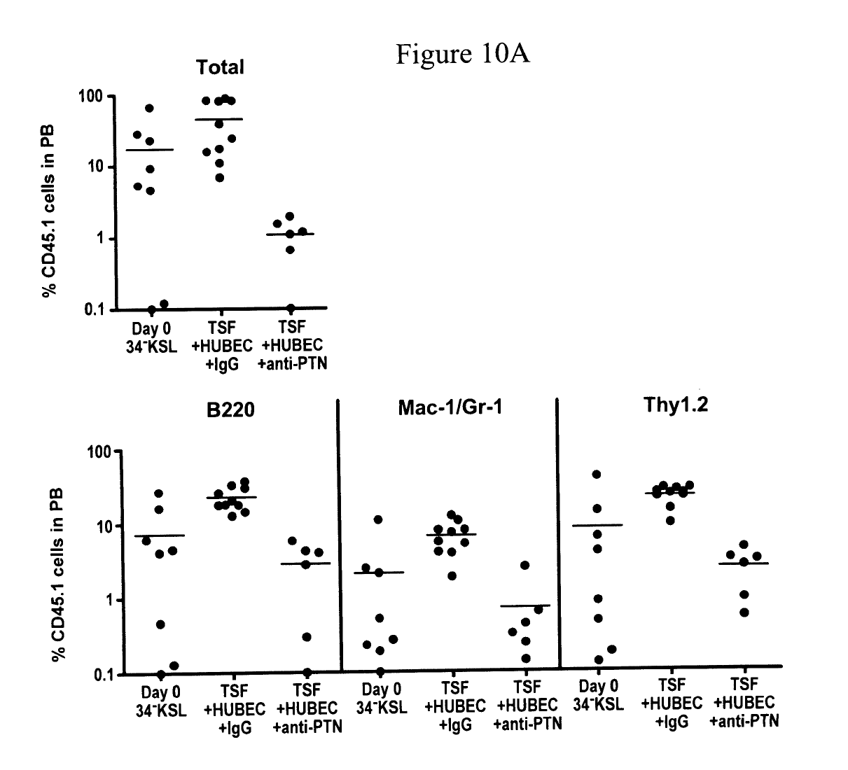

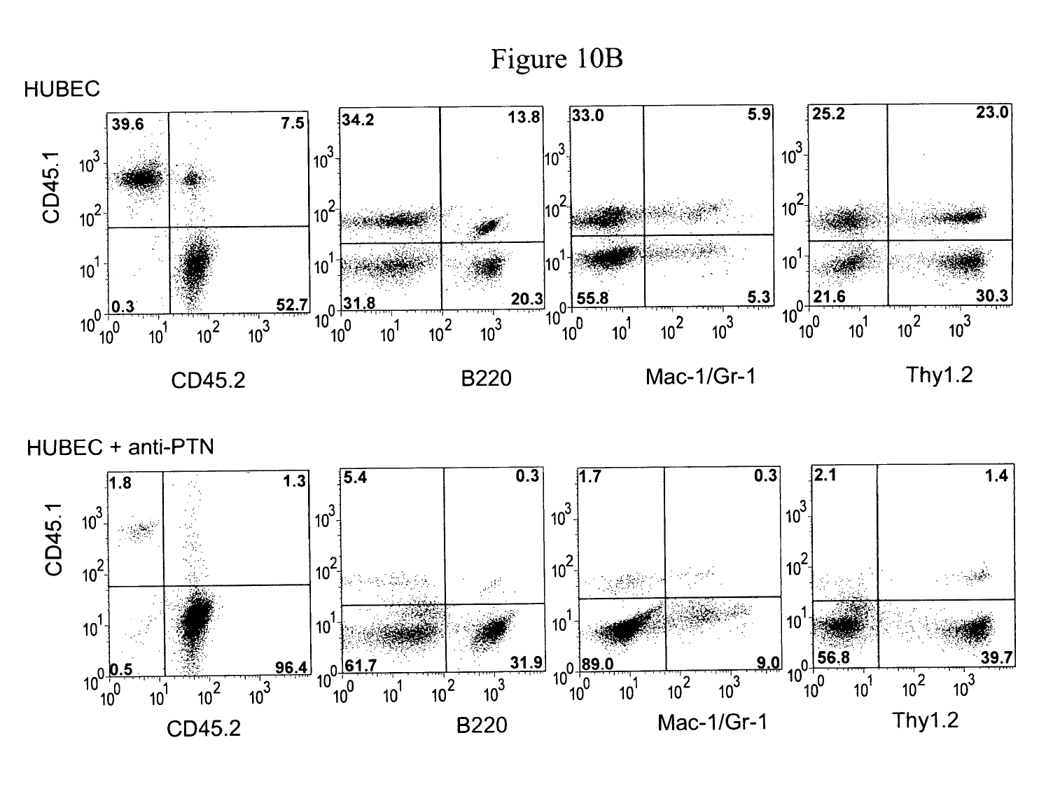

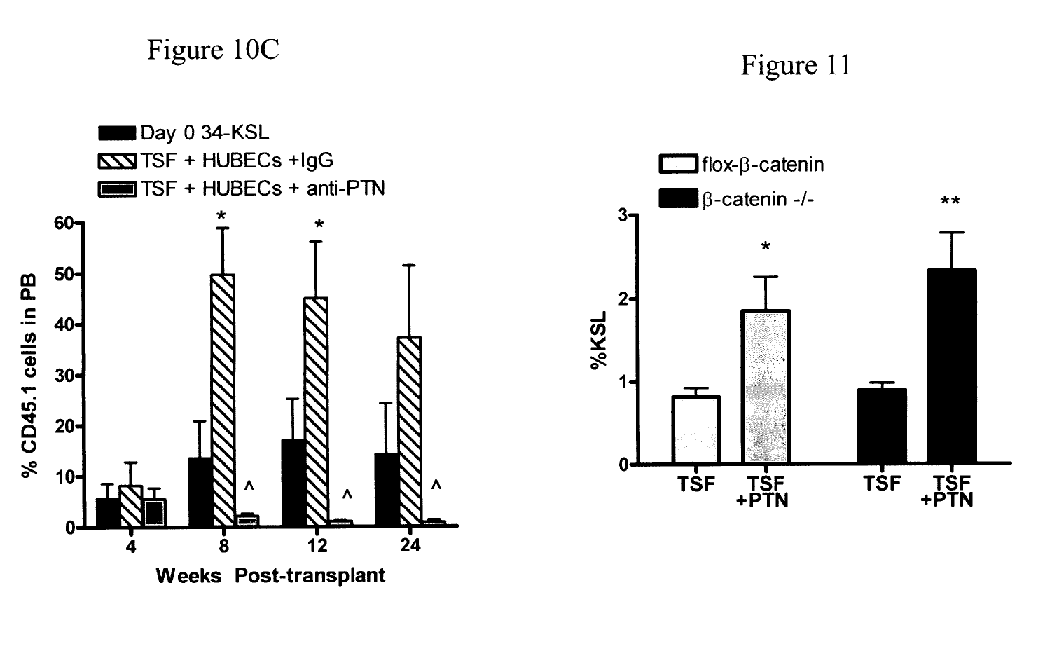

In order to further test the function of PTN in amplifying BM HSCs, an examination was made as to whether targeted inhibition of PTN signaling could block EC-mediated HSC expansion in vitro. C57Bl6 BM 34⁻KSL cells were placed in non-contact culture with HUBECs+TSF×7 days and treated with a blocking anti-PTN antibody (50 μg/mL) or isotype IgG. Competitive repopulating assays were performed with either day 0 34⁻KSL cells or their progeny following culture with HUBECs+TSF or HUBECs+TSF+anti-PTN to compare the HSC frequency within each group. C57Bl6 (CD45. 2⁺) mice that were transplanted with the progeny of 30 34⁻KSL (CD45. 1⁺) BM cells following culture with HUBECs+TSF demonstrated approximately 3-fold higher levels of donor CD45. 1⁺ cell repopulation in the PB at 12 weeks post-transplant compared to mice transplanted with the same dose of day 0 CD34⁻KSL cells (mean 45.2% vs. 17.2%, P=0.03, FIG. 10A). Conversely, mice transplanted with the progeny of the identical dose of 34⁻KSL cells following culture with HUBECs+TSF+anti-PTN demonstrated a pronounced reduction in donor CD45. 1⁺ cell and multilineage repopulation at 12 weeks (mean 1.1% vs. 45.2%, P=0.004) and through 24 weeks compared to mice transplanted with the progeny of HUBECs+TSF cultures (FIG. 10A-10C). Poisson statistical analysis from n=81 transplanted mice demonstrated that the 12 week CRU frequency in the progeny of HUBECs+TSF was 1 in 6 cells (95% CI: ⅓- 1/13) compared to 1 in 19 for day 0 34⁻KSL BM cells (95% CI: 1/10- 1/35). In contrast, the CRU frequency within the progeny of HUBECs+TSF+anti-PTN was 1 in 41 cells (CI: 1/23- 1/87), demonstrating a 7-fold reduction in HSC content in response to inhibition of PTN signaling.

### PTN Mediated Expansion of Phenotypic HSCs is Dependent Upon PI3-Kinase/Akt Signaling

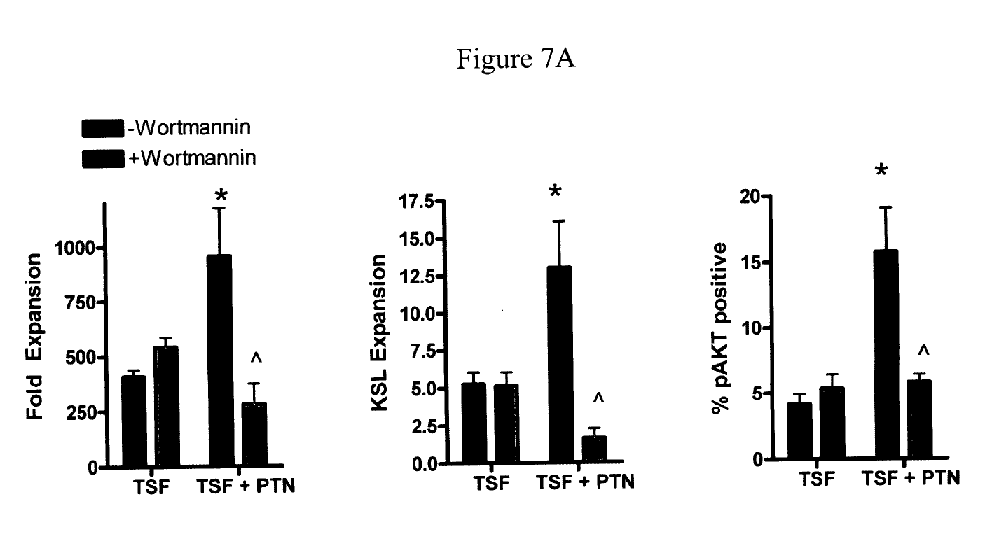

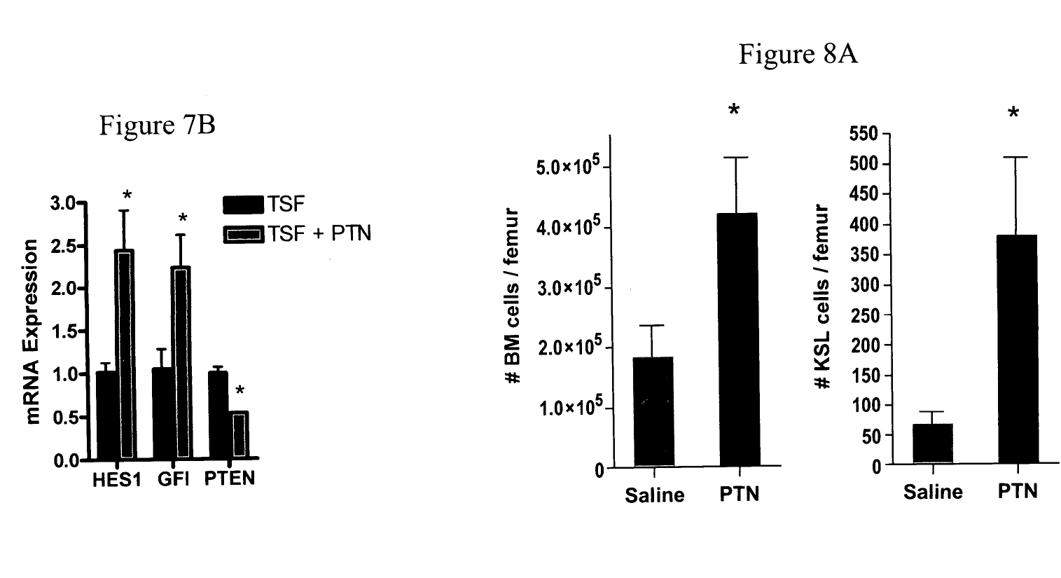

In order to determine a potential mechanism through which PTN mediates HSC expansion, an examination was made as to whether PTN altered pathways that are known to be affected by RPTIβ/ζ (Meng et al, Proc Natl Acad Sci USA 97: 2603-8 (2000), Stoica et al, J Biol Chem 276:16772-9 (2001), Landgraf et al, J Biol Chem 283:25036-45 (2008), Deuel et al, Arch Biochem Biophys 397:162-71 (2002)). Canonical PTN signaling occurs via binding and inactivation of RPTPβ/ζ (Meng et al, Proc Natl Acad Sci USA 97: 2603-8 (2000)), which can facilitate the tyrosine phosphorylation of several intracellular substrates, including Akt and β-catenin (Souttou et al, J Biol Chem 272:19588-93 (1997), Gu et al, FEBS Lett 581:382-8 (2007)). Since PTN has been shown to mediate mitogenic effects outside the hematopoietic system via activation of the PI 3-kinase/Akt pathway (Souttou et al, J Biol Chem 272:19588-93 (1997)), a test was made as to whether PTN-induced HSC amplification occurred via activation of this pathway. BM 34⁻KSL cells were treated with TSF with and without 100 ng/mL PTN in the presence and absence of 10 μM wortmannin, a PI 3-kinase inhibitor (Souttou et al, J Biol Chem 272:19588-93 (1997)). The addition of wortmannin to TSF+PTN caused a 3.4-fold reduction in total cell expansion and an 8.1-fold reduction in BM KSL cell expansion compared to cultures with TSF+PTN alone (P=0.02 and P=0.02, respectively; FIG. 7A). BM HSCs treated with TSF plus PTN demonstrated a 3.8-fold increase in levels of phosphorylated Akt, the target of PI 3-kinase, compared to treatment with TSF alone (P=0.03); the addition of wortmannin abolished this effect of PTN treatment on phosphorylated Akt levels in HSCs (P=0.04, FIG. 7A). These data confirmed that the PI 3-kinase/Akt signaling pathway was involved in mediating PTN-induced proliferation of HSCs and suggested that activation of the PI 3-k/Akt pathway contributed to the HSC expansion observed. Interestingly, PTN treatment induced the upregulation of 2 other modulators of HSC self-renewal, HES-1 and GFI-1 (Kunisato et al., Blood 101:1777-83 (2003), Hock et a, Nature 431:1002-7 (2004)) (FIG. 7B). Since HES-1, which mediates Notch signaling, has been shown to induce PI 3-kinase/Akt signaling in leukemogenesis (Palomero et al, Cell Cycle 7:965-70 (2008)), this raises the possibility that PTN induces the amplification of HSCs via induction of HES-1 and downstream activation of PI 3-kinase/Akt signaling. Consistent with this model, it was found that the expression of PTEN, a negative regulator of PTN and the PI 3-kinase/Akt pathway (Carracedo et al, Oncogene 27:5527-41 (2008)), was down-modulated in HSCs following PTN exposure. Of note, 34⁻KSL cells treated with PTN showed no increase in the activated form of β-catenin (data not shown), which is a downstream target of RPTPβ/ζ and a positive regulator of HSC self-renewal. Furthermore, when BM KSL cells from β-catenin (LoxP,LoxP;Vav-cre) mice were treated with TSF+PTN, no difference in the amplification of BM KSL cells in culture was observed between BM KSL cells from β-catenin^{-/-} mice versus cells from wild type animals (FIG. 11). Taken together, these data suggest that activation of the PI 3-kinase/Akt pathway plays an important role in mediating PTN-induced HSC expansion and these effects may be mediated by induction of HES-1.

### Systemic Administration of PTN Induces HSC Regeneration In Vivo

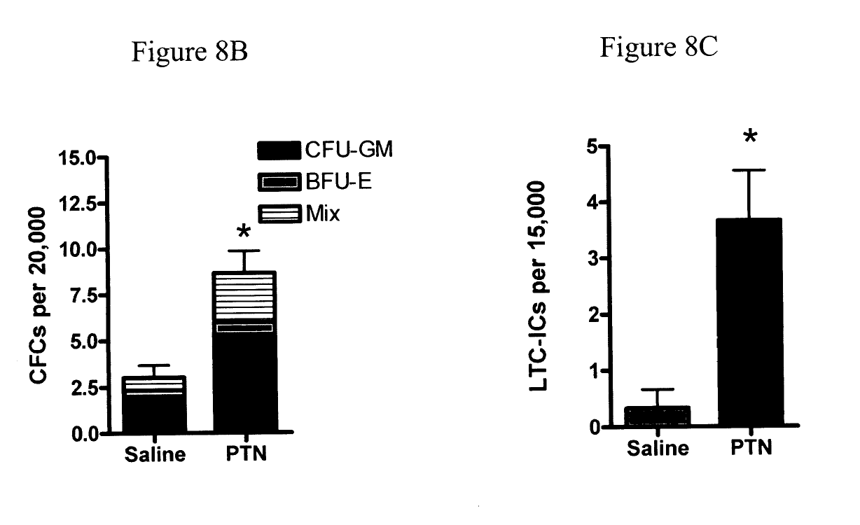

Since the addition of PTN was sufficient to induce HSC amplification in vitro a test was made as to whether administration of PTN could augment BM HSC regeneration in vivo following injury. For these experiments, mice were irradiated with 700 cGy TBI, which have been shown to cause a >20-fold reduction in BM HSC content (Salter et al, Blood 113:2104-7 (2009)), and then received 2 μg PTN or saline intraperitoneally daily×7 days. Interestingly, PTN administration caused a 2.3-fold increase in total BM cells (P=0.02) and a 5.6-fold increase in primitive BM KSL cells (P=0.02) at day +7 compared to controls (FIG. 8A). PTN treatment similarly caused a significant increase in the functional BM stem/progenitor cell pool as evidenced by a 2.9-fold increase in BM colony forming cells (CFCs) and, importantly, an 11-fold increase in long-term culture-initiating cells (LTC-ICs), which are enriched for HSCs (P=0.003 and P=0.003, respectively; FIGS. 8B and 8C). These results demonstrate that systemic treatment with PTN induces the regeneration of BM HSCs and hematopoiesis in vivo following injury.

In summary, PTN is an 18-kD heparin binding growth factor which is mitogenic for neurons (Meng et al, Proc Natl Acad Sci USA 97: 2603-8 (2000), Stoica et al, J Biol Chem 276:16772-9 (2001), Landgraf et al, J Biol Chem 283:25036-45 (2008)), has angiogenic activity (Perez-Pinera et al, Curr Opin Hematol 15:210-4 (2008), Yeh et al, J Neurosci 18:3699-707 (1998)), can function as a proto-oncogene (Chang et al, Proc Natl Acad Sci USA 104:10888-93 (2007)), but has no previously described role in hematopoiesis. The foregoing results demonstrate that PTN is a secreted growth factor for HSCs and the addition of PTN is sufficient to induce a potent expansion of LT-HSCs as demonstrated in primary and secondary competitive repopulating assays. In addition, it is shown that systemic administration of PTN causes an 11-fold expansion of BM HSCs in vivo following total body irradiation. Therefore, PTN is not only a soluble regulator of HSC selfrenewal but also HSC regeneration, a process that is largely uncharacterized. Since BM HSCs express RPTPβ/ζ and the in vitro studies demonstrate a direct effect of PTN on HSCs, it is proposed that PTN acts directly upon BM HSCs to induce BM HSC regeneration in vivo. However, it will be important to examine the effects of PTN administration on the BM microenvironment. PTN has been shown to have angiogenic activity (Perez-Pinera et al, Curr Opin Hematol 15:210-4 (2008), Yeh et al, J Neurosci 18:3699-707 (1998)) and it has been demonstrated that BM vascular endothelial cells can regulate hematopoietic reconstitution following injury (Salter et al, Blood 113:2104-7, (2009), Hooper et al, Cell Stem Cell 4:263-74 (2009)). Therefore, it is plausible that PTN might contribute indirectly to BM HSC regeneration via augmentation of BM vascular recovery. Since little is known about the extrinsic or microenvironmental signals that regulate BM HSC regeneration in vivo (Congdon et al, Stem Cells 26:1202-10 (2008)), the demonstration that PTN induces BM HSC regeneration in vivo has fundamental importance toward understanding this process. Furthermore, since PTN is a soluble growth factor capable of inducing BM HSC regeneration in vivo, it is unique compared to previously described methods to induce HSC self-renewal (Reya, et al, Nature 423:409-14 (2003), Hackney et al, Proc Natl Acad Sci USA 99:13061-6 (2002), Antonchuk et al, Cell 109:39-45 (2002)).

It is also shown that PTN induces PI3-kinase/Akt signaling in BM HSCs and inhibition of PI3-kinase/Akt signaling blocked PTN-induced proliferation and expansion of BM KSL cells in culture. PTN also induced the expression of HES-1, a downstream mediator of Notch signaling and a positive regulator of PI3-kinase/Akt signaling (Kunisato et al., Blood 101:1777-83 (2003), Palomero et al, Cell Cycle 7:965-70 (2008)), suggesting the possibility that PTN induces HSC amplification via activation of Notch signaling. Conversely, Zhang et al. recently reported that deletion of PTEN, a negative regulator of PI3-kinase/Akt signaling, was associated with exhaustion of 12 week CRU in mice (Zhang et al, Nature 441:518-22 (2006)); in addition, deletion of FoxO3a, a transcription factor which negatively regulates HSC cycling and is inhibited by Akt, has been associated with depletion of LT-HSCs in mice (Miyamoto et al, Cell Stem Cell 1:101-12 (2007)). Therefore, it will be important to confirm whether PTN-mediated expansion of HSCs is dependent upon PI3-kinase/Akt signaling or whether PTN-mediated HSC expansion is a function of alternative self-renewal pathways (e.g. Notch signaling via HES-1 induction).

Research in stem cell biology has yielded much information about the intrinsic and extrinsic pathways that regulate HSC self-renewal and differentiation (Zon, Nature 453: 306-13 (2008), Orkin et al, SnapShot: hematopoiesis. Cell 132:712 (2008), Kiel et al, Nat Rev Immunol 8:290-301 (2008), Blank et al, Blood 111:492-503 (2008), Adams et al, Nat Biotechnol 25:238-243 (2007)). However, the successful development of soluble growth factors or cytokines capable of inducing HSCs expansion ex vivo or HSC regeneration in vivo has remained an elusive goal (Blank et al, Blood 111:492-503 (2008), Adams et al, Nat Biotechnol 25:238-243 (2007)). Here, it is shown that PTN is a soluble growth factor for HSCs which induces LT-HSC expansion ex vivo and HSC regeneration in vivo following injury. PTN therefore has unique potential for the expansion of human HSCs ex vivo and to induce hematopoietic regeneration in patients following myelotoxic chemo- and radiotherapy.

### Example 3

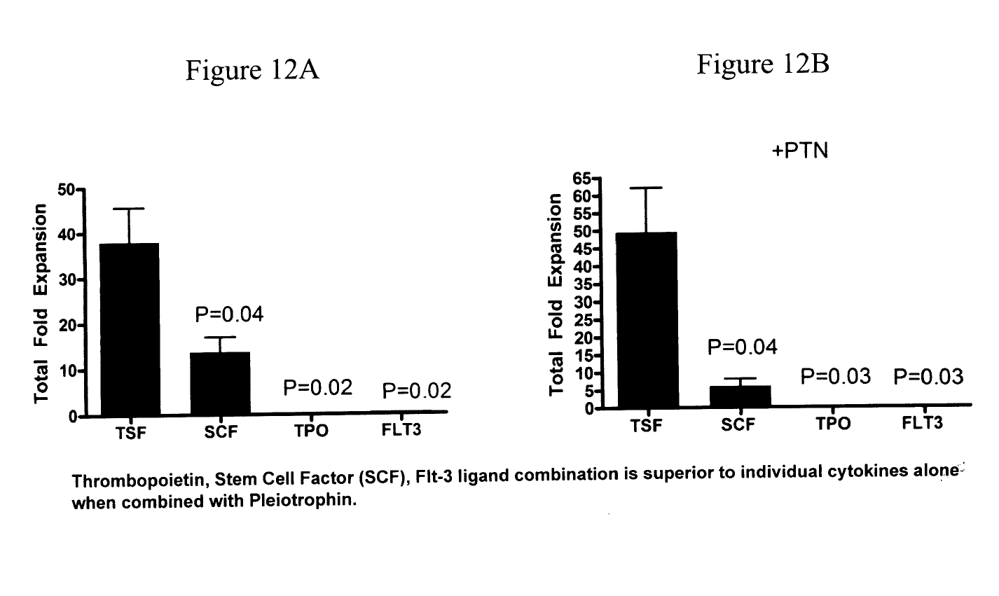

Bone marrow lineage negative (lin-) progenitor cells were placed in culture for 7 days with 20 ng/mL thromobopoietin (TPO), 120 ng/mL stem cell factor (SCF) or 50 ng/mL Flt-3 ligand or the combination of all 3 cytokines (TSF) with and without 100 ng/mL pleiotrophin (PTN). Neither thromobopoietin alone nor Flt-3 ligand alone supported viable BM progenitor cells in culture (FIG. 12A). SCF+/−PTN supported a modest expansion of BM progenitor cells in culture but the combination of TSF was superior to all individual cytokines tested in expanding BM progenitor cells (P=0.04, 0.02, 0.02) and this 37-fold expansion was increased to 49-fold when TSF was combined with PTN (FIG. 12B). Taken together, these data demonstrate that TSF+PTN is the optimal combination to expand hematopoietic progenitor cells in culture.

All documents and other information sources cited above are hereby incorporated in their entirety by reference.

TABLE 1
Genes overexpressed by HUBECs

| Fold | | |
|---|---|---|
| Change | Symbol | Name |
| 75.22 | SCG2 | secretogranin II (chromogranin C) |
| 43.29 | IGFBP1 | insulin-like growth factor binding protein 1 |
| 25.61 | APOE | apolipoprotein E |
| 25.06 | PTN | pleiotrophin |
| 23.29 | CX3CL1 | chemokine (C-X3-C motif) ligand 1 or fractalkine |
| 17.75 | OLFML2A | olfactomedin-like 2A |
| 16.42 | TNFRSF11B | tumor necrosis factor receptor superfamily, member 11b (osteoprotegerin) |
| 16.09 | HAPO | hemangiopoietin |
| 13.23 | LGALS3BP | lectin, galactoside-binding, soluble, 3 binding protein |
| 12.1 | CXCL12 | stromal cell-derived factor 1 |
| 11.43 | IGFBP2 | insulin-like growth factor binding protein 2, 36 kDa |
| 7.837 | IGFBP3 | insulin-like growth factor binding protein 3 |
| 5.714 | SEMA3B | sema domain, immunoglobulin domain, secreted, (semaphorin) 3B |

TABLE 2
CRU frequencies in BM 34⁻KSL cells and their progeny

| | | No. | CRU | 95% Confidence |
|---|---|---|---|---|
| BM source | Cell Dose | Engrafted | Estimate | Interval |
| Day 0 | 10 | 0 of 9 | 1 in 39 | 1/21-1/70 |
| 34⁻KSL | 30 | 6 of 10 | | |
| | 100 | 7 of 7 | | |
| TSF | 10 | 2 of 9 | 1 in 58 | 1/31-1/108 |
| | 30 | 1 of 6 | | |
| | 100 | 8 of 9 | | |
| TSF + PTN | 10 | 6 of 8 | 1 in 10 | 1/5-1/20 |
| | 30 | 7 of 8 | | |
| | 100 | 9 of 9 | | |

BM 34⁻KSL cells (CD45.1⁺) or their progeny following 7 day culture were transplanted at limiting dilutions into lethally irradiated C57BI6 (CD45.2⁺) mice along with 1 × 10⁵ host BM MNCs in a competitive repopulating assay.
At 12 weeks post-transplant, PB was collected from all recipient mice and flow cytometric analysis was performed to measure CD45.1⁺ donor-derived cell repopulation in the recipient mice.
Positive engraftment was defined as ≧1% CD45.1⁺ cells in the recipient mice.
Poisson statistical analysis using the maximum likelihood estimator was performed to estimate the CRU frequency in each group^{20,37}.

What is claimed is:

1. A method of enhancing the expansion of hematopoietic stem cells (HSCs) in vitro comprising culturing HSCs in the presence of an amount of pleiotrophin (PTN) sufficient to enhance said expansion.

2. The method according to claim 1 wherein said method comprises culturing said HSCs in the presence of PTN and at least one of thrombopoietin, stem cell factor (SCF) and Flt-3 ligand.

3. The method according to claim 2 wherein said method comprises culturing said HSCs in the presence of PTN, thrombopoietin, SCF and Flt-3 ligand.

4. The method according to claim 1 wherein said HSCs are mammalian HSCs.

5. The method according to claim 4 wherein said mammalian HSCs are human HSCs.

6. The method according to claim 1 wherein said HSCs are derived from umbilical cord blood.

7. The method according to claim 1 wherein said PTN is human PTN.

8. The method according to claim 1 wherein said PTN is recombinant mammal PTN.

9. A method of restoring hematopoietic function comprising administering to a mammalian subject in need thereof HSCs expanded in vitro in the presence of PTN, wherein said HSCs are administered in an amount sufficient to restore said function.

10. The method according to claim 9 wherein said subject is a human subject.

11. The method according to claim 9 wherein said HSCs are autologous HSCs.

12. The method according to claim 9 wherein said expanded HSCs are administered to said subject to accelerate hematologic recovery following chemo- or radiation-therapy.

13. The method according to claim 12 wherein said method comprises:
i) obtaining a marrow sample from said subject prior to said chemo- or radiation therapy,
ii) expanding HSCs from said marrow sample in the presence of PTN, and
iii) administering said expanded HSCs to said subject following said chemo- or radiation-therapy so that said hematologic recovery is accelerated.

14. The method according to claim 9 wherein said HSCs are expanded in the presence of PTN, and at least one of thrombopoietin, SCF and Flt-3 ligand.

15. The method according to claim 14 wherein said HSCs are expanded in the presence of PTN, thrombopoietin, SCF and Flt-3 ligand.

16. A method of restoring hematopoietic function comprising administering to a mammalian subject in need thereof an amount of PTN sufficient to restore said function.

17. The method according to claim 16 wherein said subject is a human and said PTN is human PTN.

18. The method according to claim 16 wherein an expression construct comprising a sequence encoding PTN is administered under conditions such that said sequence is expressed and said hematopoietic function is restored.

19. The method according to claim 18 wherein said sequence is operably linked to a promoter.

20. The method according to claim 18 wherein said sequence is present in a viral vector.

21. A method of stimulating hematopoietic recovery in a mammal following chemotherapy or radiotherapy comprising administering to said mammal an amount of PTN sufficient to effect said stimulation.

22. The method according to claim 21 wherein said PTN is administered subcutaneously or intraperitoneally.

23. A method of accelerating hematologic recovery in a mammal following chemotherapy or radiotherapy comprising administering to said mammal PTN and granulocyte colony stimulating factor in an amount sufficient to effect said acceleration.
